(19) United States
(12) Patent Application Publication
(10) Pub. No.: US 2023/0029766 A1
(43) Pub. Date: Feb. 2, 2023

(54) REGISTERING HUE AND/OR COLOR FOR NON-INVASIVE TISSUE SURFACE ANALYSIS

(71) Applicant: RHODE ISLAND HOSPITAL, Providence, RI (US)

(72) Inventors: Gregory D. JAY, Providence, RI (US); Selim SUNER, Providence, RI (US); James RAYNER, Providence, RI (US)

(21) Appl. No.: 17/789,740

(22) PCT Filed: Dec. 30, 2020
(86) PCT No.: PCT/US2020/067456

§ 371 Date: Jun. 28, 2022

Related U.S. Application Data
(60) Provisional application No. 62/955,787, filed on Dec. 31, 2019.

(51) Int. Cl. A61B 5/145; A61B 5/1455; A61B 5/00; A61B 5/103; G06T 7/00; G06T 7/11; G06V 10/766

(52) U.S. Cl. CPC A61B 5/14546; A61B 5/0077; A61B 5/1032; A61B 5/1455; A61B 5/6898; A61B 5/7264; A61B 5/7275; G06T 7/11; G06T 7/0012; G06V 10/766; G06T 2207/10024; G06T 2207/20081; G06T 2207/20084; G06T 2207/30041

(57) ABSTRACT

The current subject matter can include non-invasive systems and methods for detecting hemoglobin. In one implementation, a method includes receiving data characterizing a plurality of images associated with palpebral conjunctiva region of one or more patients. The method also includes receiving data indicative of a user selection of a first pixel of a first image of the plurality of images. The method further includes determining, a region of interest associated with the selected first pixel. The determining can include identifying a plurality of pixels adjacent to the first pixel having color parameter values within a predetermined range of a first color parameter value of the first pixel. The method also includes determining a first plurality of parameters associated with the region of interest, and providing the first plurality of parameters.

### RELATED APPLICATION

This application claims priority under 35 U.S.C. § 119(e) to U.S. Provisional Pat. Application Number 62/955,787 filed on Dec. 31, 2019, the entire content of which is hereby expressly incorporated by reference herein.

### TECHNICAL FIELD

The current subject matter relates to methods and systems for detection of hemoglobin (Hb or HB) concentration in blood.

### BACKGROUND

Blood tests are used in health care to determine physiological and biochemical states, such as disease, mineral and organic molecule content, pharmaceutical drug effectiveness, and organ function. Blood test can involve extraction of blood sample (e.g., from a vein). This determination can be achieved through invasive techniques using a hypodermic needle or a finger prick. Detection of specific blood components can be grouped together into one test panel (or “blood panel”). For example, a complete blood count (CBC) test can also be used to detect hemoglobin concentration, the oxygen-carrying protein in red blood cells.

### SUMMARY

Anemia, defined as a low hemoglobin concentration, is a disorder that has deleterious impacts on health and well-being of humans and mammals in general. Anemia can have a direct impact on morbidity and mortality, can exacerbate many health conditions such as cardiovascular disease, and can impact the economy by reducing worker productivity. Hemoglobin (Hb) can be clinically measured by the complete blood count (CBC) that requires obtaining blood and acquisition of expensive devices. The current subject matter represents an improvement over existing methods by utilizing non-invasive systems and methods for detecting Hb.

In an aspect, a method includes receiving data characterizing a plurality of images associated with palpebral conjunctiva region of one or more mammals (e.g., humans, patients, etc.). The method also includes receiving data indicative of a user selection of a first pixel of a first image of the plurality of images, the first pixel depicting a representation of the conjunctiva color. The method further includes determining, a region of interest associated with the selected first pixel. The determining can include identifying a plurality of pixels adjacent to the first pixel having color parameter values within a predetermined range of a first color parameter value of the first pixel. The method also includes determining a first plurality of parameters associated with the region of interest, and providing the first plurality of parameters.

One or more of the following features can be included in any feasible combination. For example, in some implementations, the method can further include generating a matrix comprising a first plurality of rows and a second plurality of columns. The first plurality of rows are representative of the plurality of images and the second plurality of columns are representative of the first plurality of parameters. In some implementations, a predictive model for Hb is generated by performing a regression analyses (e.g., linear regression) which can include complex machine learning and neural network paradigms, on the generated matrix. In some implementations, the method further includes manipulating the received data characterizing the plurality of images. The manipulating can include generating a dataset by mapping one or more values of the received data based on a predetermined look-up table.

In some implementations, the method further includes presenting the first image on a graphical user interface display space. In some implementations, data indicative of user interaction with the first pixel is based on a user interaction with the first image in the graphical user interface display space. In some implementations, the method can further include storing the selected region in a database.

In some implementations, the data characterizing the plurality of images include unprocessed and uncompressed sensor data. In some implementations, determining the first plurality of parameters does not include determining hyperspectral data based on the received data characterizing the plurality of images. In some implementations, determining the first plurality of parameters does not include determining a spectral-super resolution spectroscopy model from the unprocessed and uncompressed sensor data. In some implementations, determining the first plurality of parameters does not include solving for multiple wavelengths with the visible light band that were not acquired by an image sensor. In some implementations, the predictive model for Hb is not trained on hyperspectral data.

Non-transitory computer program products (i.e., physically embodied computer program products) are also described that store instructions, which when executed by one or more data processors of one or more computing systems, causes at least one data processor to perform operations herein. Similarly, computer systems are also described that may include one or more data processors and memory coupled to the one or more data processors. The memory may temporarily or permanently store instructions and information (e.g., look-up tables) that can cause at least one processor to perform one or more of the operations described herein. In addition, methods can be implemented by one or more data processors either within a single computing system or distributed among two or more computing systems. Such computing systems can be connected and can exchange data and/or commands or other instructions or the like via one or more connections, including a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

FIG. 1 is a flowchart illustrating an exemplary method of Hb detection;

FIG. 2 is a bar graph illustrating an exemplary distribution of Hb concentration of a group of patients;

FIG. 3 is a series of photographs illustrating an exemplary selection of a conjunctiva region;

FIG. 4 is a series of line graphs illustrating correlation between Hb obtained using this algorithm from images (IHB) and Hb obtained from the gold standard blood test (CBC HB);

FIG. 5 is a dot plot illustrating error in calculation of IHB relative to CBC HB in a predictive modeling set;

FIG. 6 is a dot plot illustrating an exemplary Bland-Altman plot of right and left averaged IHB versus CBC HB in a predictive modeling set;

FIG. 7 is a line graph illustrating the tradeoff of between sensitivity and specificity of a classical receiver operator curve in a predictive modeling set;

FIG. 8 illustrates an exemplary image of conjunctival anatomy;

FIG. 9 illustrates and exemplary distribution of Hb concentration;

FIG. 10 illustrates and exemplary distribution of Massey Scores;

FIG. 11 illustrates an exemplary Observer Image Quality score distribution;

FIG. 12 illustrates exemplary correlation of HBc and HBl between images of the right and left eye in a predictive modeling set;

FIG. 13 illustrates an exemplary Bland-Altman plot for HBc compared to HBl in a validation modeling set;

FIG. 14 illustrates exemplary Receiver Operator Characteristics of HBc using HBl as the gold standard in a validation modeling set;

FIG. 15 illustrates an exemplary Bland-Altman plot for HBc compared to HBl in a validation modeling set for different segments of data quality;

FIG. 16 illustrates another exemplary Bland-Altman plot for HBc compared to HBl for different segments of Massey score;

FIG. 17 illustrates an exemplary method of non-invasive measurement of Hb;

FIG. 18 illustrates another exemplary method of non-invasive measurement of Hb;

FIG. 19 illustrates an exemplary representation of three N x M matrices where each matrix represents the red, green, and blue components of an RGB color image;

FIG. 20 illustrates an exemplary selection of a seed point on conjunctiva via a graphical user interface (GUI);

FIG. 21 illustrates an exemplary GUI display space for uploading RAW images and selection of seed point;

FIG. 22 illustrates an exemplary plot of predicted vs actual Hb measurement;

FIG. 23 illustrates an exemplary application installed on an iPhone for non-invasive detection of Hb;

FIG. 24 illustrates and exemplary GUI of the application in FIG. 23 for capturing an image of an eye; and

FIG. 25 illustrates an exemplary file folder for storing data for the application in FIG. 23.

### DETAILED DESCRIPTION

Anemia, defined as a low Hb concentration, is a disorder that has deleterious impacts on health and wellbeing of humans and mammals in general. Anemia can have a direct impact on morbidity and mortality, can exacerbate many health conditions such as cardiovascular disease, and can impact the economy by reducing worker productivity. Hb can be clinically measured by the complete blood count (CBC) that requires obtaining blood and acquisition of expensive tools.

Traditional techniques of detecting Hb (e.g., CBC) can be invasive (e.g., require extraction of blood). These techniques can require infrastructure (e.g., laboratory, biochemical reagent, running water, electricity, etc.), work force (e.g., trained technicians) that can be expensive, require a finite amount of time and be logistically unfeasible. Some implementations of the current subject matter include non-invasive systems and methods of detecting Hb based on digital images (e.g., of palpebral conjunctiva) that can be obtained, for example, from a mobile device, such as a smartphone. In some implementations, the smartphone can capture transmitted and/or reflected light signals, process digital images (e.g., of palpebral conjunctiva) to predict Hb concentration and screen for anemia. These techniques can be non-invasive and inexpensive, and can be suitable for diagnosing anemia in areas with limited access to healthcare infrastructure, or when results are required instantly; a significant advantage over other methods of determining Hb concentration. Some implementations of the current subject matter can include an interactive application that can allow a user to take pictures of the conjunctiva (e.g., inner layer of eyelids) and execute an image analysis technique (e.g., an image analysis algorithm executed on the smartphone). The image analysis algorithm can process the raw images captured by the camera to improve the quality of the captured image presented to the user. For example, the RAW images can provide data directly from the camera sensor without the typical processing and compression that occurs with typical images. The image analysis algorithm can be processed in real-time to generate a processed image (based on processed RAW image data). For example, the unprocessed sensor data can be linearized or otherwise transformed (e.g., based on a look-up table associated with the sensor that generated the unprocessed sensor data). Transformation can include mapping each data value in the unprocessed data based on the look-up table. The image analysis algorithm can account for white balance, ambient lighting, glare, pigmentation of the surrounding skin, and detect borders of conjunctiva separated by other anatomical features in the image such as the sclera (white), pupil (black), edges of the eyelid, and the like. Processing of the raw image can enable improved detection of Hb.

Anemia is a condition in which the blood Hb concentration is below a desirable Hb value. The Hb values which define anemia are set by the World Health Organization (WHO) and are different for males, females and children. The symptoms of anemia (e.g., fatigue, dizziness, headache, shortness of breath, difficulty concentrating, etc.) can lead to major social and economic consequences such as lost wages and expensive medical care [Smith RE (March 2010). “The clinical and economic burden of anemia”. The American Journal of Managed Care. 16 Suppl Issues: S59-66. PMID 20297873.] in the long term. Anemia can also be a life-threatening condition. Severe anemia can be a sequela of occult blood loss, malnutrition or underlying disease, and can be a significant risk factor for morbidity and mortality (e.g., in vulnerable populations such as children, the elderly and the chronically ill [S. P. Scott, L.P. Chen-Edinboro, L. E. Caulfield, L. E. Murray-Kolb. Nutrients. 2014 Dec, 5915-5932, S. D. Denny, M. N. Kuchibhatla, H. J. Cohen. Am J Med. 2006 Apr, 327-34]). Anemia is widely prevalent, affecting an estimated 5.6% of Americans and more than 25% of the global population [de Benoist B et al., eds. Worldwide prevalence of anaemia 1993-2005. WHO Global Database on Anaemia Geneva, World Health Organization, 2008.

The clinically used gold standard test for diagnosis is the complete blood count (CBC), which requires trained phlebotomists and laboratory technicians and the use of biochemical reagents and expensive lab equipment [E. L. Gottfried. N Engl J Med. 1979 May 31,1277, A. Karnad, T. Poskitt. Arch. Intern. Med. 1985, 1270-1272] as well as requiring time to transport the sample to point of testing and process and return a result. In many rural settings with little access to healthcare, screening for or diagnosing anemia with a CBC may not be economically or logistically feasible. Prevalence of anemia in rural populations reflects the social determinants of health and the adverse effects of living in resource poor communities [World Health Organization. WHO, Geneva, 2008]. Also, when time is critical such as in severe trauma, knowing the Hb concentration rapidly may be life-saving. There is an unmet need for inexpensive, accessible and non-invasive tools capable of screening and diagnosing anemia.

Cost can be an obstacle to widespread adaptability of novel, non-invasive technology. The fixed cost of implementing a spectroscopic or a retinoscopic device is prohibitive for many rural and urban communities and individuals. Ideally, a desirable screening method can be based on a pre-existing technology that is widely prevalent. An estimated 2.7 billion people, or 36% of the world’s population, used smartphones in 2019. This number is expected to grow to 2.87 billion by 2020 [Cisco. https://www.cisco.com/c/en/us/solutions/collateral/service-provider/visual-networking-index-vni/white-paper-c11-738429.html]. Smartphone ownership is growing at a rate faster than that of the global population [Cisco. https://www.cisco.com/c/en/us/solutions/collateral/service-provider/visual-networking-index-vni/white-paper-c11-738429.html]. Affluent individuals are more likely to own smartphones, but trends suggest that popularity of smartphones is growing steadily worldwide. The Middle East and Africa, who have the lowest estimated rate of smartphone use, still are projected to have 13.7% of citizens using smartphones [Cisco. https://www.cisco.com/c/en/us/solutions/collateral/service-provider/visual-networking-index-vni/white-paper-c11-738429.html].

The device and methods described herein feature non-invasive measurement of Hb concentration in human and other mammalian tissues and/or fluids. Hb is one of the strongest chromophores found in human tissue. Previous devices measure Hb using transcutaneous, retinal, and mucosal spectroscopy or photography. Each of these methods can pose challenges. For example, the wide variance in the quantity/abundance of other tissue chromophores such as bilirubin and melanin can reduce the accuracy of Hb measurement with transcutaneous spectroscopy. This challenge is amplified when considering the spectroscopic evaluation of ethnically, genetically, and physically diverse populations.

The fingernail bed, palmar creases, and conjunctiva tissues are devoid of melanocytes. Hb measurement using light-based modalities at these sites is more accurate, given the absence of a “competing” chromophore. A smartphone application has been developed to measure Hb concentration from the nailbed using digital photography and has an accuracy of ±2.4 g/dL [R.G. Menino, D.R. Myers, E.A. Tyburski, J. Boudreau, T. Leong, G.D. Clifford, W.A. Lam. Nature Communications. 2018 Dec 4;9(1):4924]. But, while promising, these results are not generalizable to cyanotic, hypotensive or mildly hypothermic patients [J. S. McGrath, S Datir, F O’Brien. BMJ Case Rep. 2016 Nov, 28].

The palpebral conjunctiva is a highly vascular mucocutaneous surface with minimal connective tissue between the outer mucous membrane (which is devoid of any chromophores) and blood vessels. In addition to the absence of melanin, there is also no epidermis, dermis or subcutaneous fat which could impede the transmission of light. These layers of tissue can be potential confounders and can make image analysis of the deeper vascular structures less accurate. Conjunctival pallor can be a sign of severe anemia on physical exam. However, the sensitivity of this finding can be highly clinician dependent, with poor inter-observer reliability [T.N. Sheth, BArtsSc, N.K. Choudhry, M. Bowes, A.S. Detsky. J Gen Intern Med. 1997 Feb; 12(2):102-106, A. Kalantri, M. Karambelkar, R. Joshi, S. Kalantri, U. Jajoo. PLoS One. 2010;5(1)]. Utilization of this surface for sampling and analysis of digital imaging can result in sensitive and accurate screening for anemia [S. Suner, G. Crawford, J. McMurdy, G. Jay. J Emerg Med. 2007; 33(2):105-111].

In some implementations, the current subject matter can provide systems and methods of affordable and non-invasive measurement of Hb concentration using mobile device (e.g., smartphone) digital photography of the palpebral conjunctiva. This procedure can be rapid, cost effective and does not require extensive equipment infrastructure. This procedure can be achieved, for example, by eversion of the bottom eyelid for exposure of the conjunctiva, and capturing the image of the exposed conjunctiva by a user device (e.g., smartphone, tablet, etc.), by image capture minimizing motion, shadow and glare, etc. Subsequent rapid, real-time computation using an image analysis algorithm can be processed on a smartphone utilizing an on-device application. The real-time computation can account for ambient lighting, glare, pigmentation of the surrounding skin, and produce an approximate value for Hb concentration. There has been recent promising work in this area using digitized images of the conjunctiva to emulate spectra super-resolution spectroscopy for the analysis of Hb [Park SM, Visbal-Onufrak MA, Haque MM, Were MC, Naanyu V, Hasan MK, et al. mHealth spectroscopy of blood Hb with spectral super-resolution. Optica. 2020]. The procedure may require little skill to be implemented. The user device can include an interactive application that can allow for detection of Hb concentration based on the captured image and selected region for analysis. The ability to detect Hb concentration using a smartphone can allow healthcare providers or novice users to quickly and accurately screen for anemia even under austere conditions without the need for trained phlebotomists, other health care professionals or expensive laboratory equipment. This can result in elimination of wait times for results.

The interactive application can allow the user to take pictures of the conjunctiva (e.g., inner layer of eyelids). In some implementations, the application can guide the user to capture desirable images. For example, the application can provide instructions to the user to reduce (e.g., minimize) the motion of the camera capturing the image, maximize image focus and reducing shadow/glare on the exposed conjunctiva or guide them to a desirable region (e.g., optimal region) on the conjunctiva such as one containing maximal vascularity. The interactive application can be used to select a region of interest on the captured image and can be configured to execute an image analysis technique at the backend (e.g., an image analysis algorithm executed on the smartphone). The image analysis algorithm can process the RAW images captured by the camera to improve the quality of the captured image presented to the user. The image analysis algorithm can be processed in real-time to generate a processed image (based on processed RAW image data). The image analysis algorithm can account for white balance, ambient lighting, glare, pigmentation of the surrounding skin, and detect borders of conjunctiva separated by other anatomical features in the image such as the sclera (white), pupil (black), edges of the eyelid, etc.

Based on the processed RAW data, the interactive application can generate a selection of pixels within the area of interest (e.g., a vascular region of conjunctiva, devoid of light glare, eyelashes, portions of other anatomy such as sclera) of the captured image of the conjunctiva, and present the improved image to the user. This can allow the user to accurately identify a portion of the image. The interactive application can receive an input from the user indicative of the identification (e.g., based on the user touching a screen of the smartphone displaying the improved image) and identify pixels in the improved image associated with conjunctiva. In some implementations, the user interface can guide the user to select a pixel (e.g., prompt the user to zoom in and select a pixel if the initial selection is not of desirable precision). In some implementations, an automated pixel selection paradigm can be employed. The pixel selection paradigm can be implemented by an algorithm which selects neighboring pixels with similar characteristics (e.g., defined by a set of criteria) in a concentric centrifugal expansion resulting in a crystallization process until a hard edge (e.g., a priori defined using image based criteria). The criteria or set of criteria can be defined to select similar pixels with respect to the initial user-selected pixel. In some implementations, pixel similarity can be based on one or more of red hue, red intensity, other color parameters, borders and edges, etc. [Yi-Ming Chem et al. Examining palpebral conjunctiva for anemia assessment with image processing methods, https://pubmed.ncbi.nlm.nih.gov/28110719/]. For example, the selection criteria can specify selection of all adjacent pixels with a red hue within 75% of the initial user selected pixel until an edge is reached. Multiple or nested criteria may also be specified.

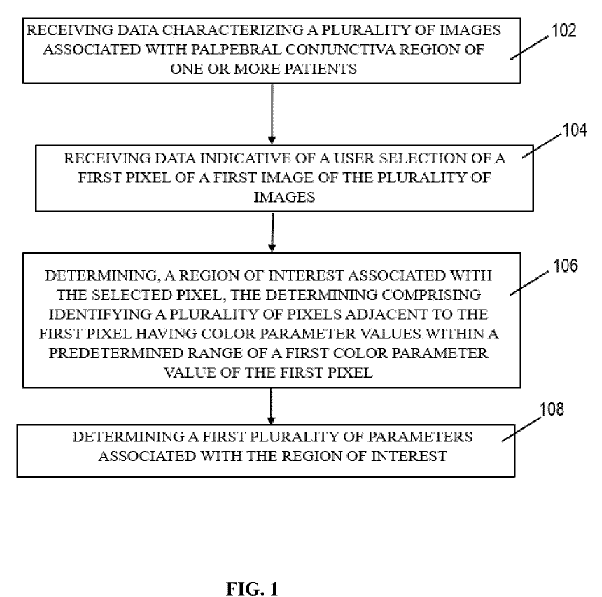

FIG. 1 illustrates an exemplary method of detecting hemoglobin. At 102, data characterizing a plurality of images associated with conjunctiva region of one or more patients is received (e.g., by a processor). The plurality of images can be received from a database of images of conjunctiva regions. The database can include additional patient information (e.g., image meta-data, blood test reports) that can be received by the processor.

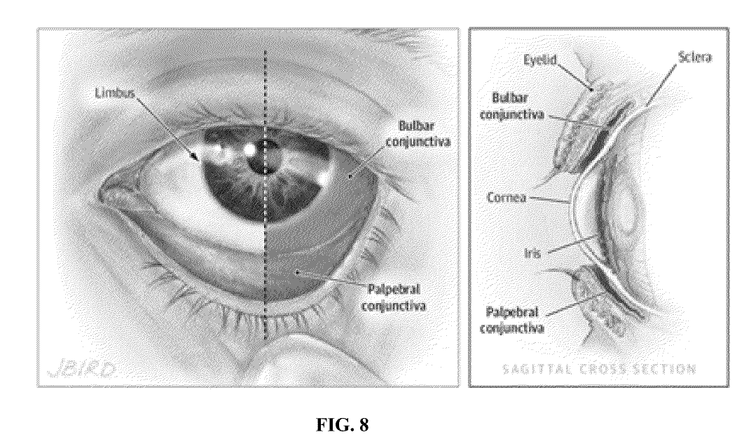

Exemplary image collection will now be described. In a sample study, images of conjunctiva regions were collected from patients who were in the emergency department at Rhode Island Hospital. A CBC was obtained as part of the patients care within 4 hours of digital image acquisition. This information was used to select images that expose the palpebral conjunctiva of both eyes of the patients (e.g., where image of each eye obtained separately). The conjunctiva is the mucous membrane which lines the inner lower eyelid. This membrane has two major components: the palpebral conjunctiva which is more vascular and adherent to the eyelid (inner surface) and the bulbar conjunctiva which is less vascular and is associated with the globe, partially overlying the sclera. The palpebral conjunctiva can be more vascular, and likely the region of the conjunctival surface with the most information regarding Hb. FIG. 8 illustrates an exemplary image of conjunctival anatomy.

Patients with injury or infection of the eye were excluded. Patients could be lying supine or in a sitting position. Images were obtained under ambient lighting in the patient’s hospital room. In the initial development phase of the study, 32 images were obtained from each patient. Eight images were obtained of the conjunctiva with a standard color reference (e.g., color checker, Passport Photo, x-rite, etc.) adjacent to each eye (e.g., one of the right eye and one of the left eye with the conjunctiva exposed). Captured images (both with and without flash) were in raw and jpeg format. The remaining 24 images were obtained without the color reference (e.g., as close to the conjunctiva as can be sharply focused). Care was taken to minimize glare from ambient light sources and minimize movement. Patients were asked to remain still and pull down their lower eyelid to expose their conjunctiva. Images were obtained using Halide (e.g., Chroma Noir LLC San Francisco, CA.) application on an iPhone 8 plus (Apple Inc, Cupertino, CA.). Demographic information including gender, age and Massey skin color rating, vital signs (e.g., blood pressure, heart rate, respiratory rate temperature, pulse oximetry, etc.), hospital laboratory reported lab tests (e.g., Hb, total and direct bilirubin if available), time of lab tests, and time of image collection were recorded on a data collection form. To reduce variability, all data were collected by one of the investigators who developed the data collection methods. In some studies, multiple images were captured in similar fashion from the same patient with a researcher and the patient themselves to compare accuracy. The Hb distribution was plotted weekly and patient selection was adjusted to maximize a wide distribution of Hb values.

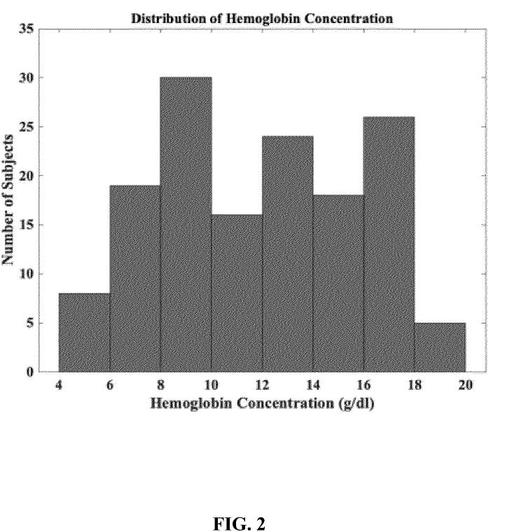

FIG. 2 illustrates the distribution of Hb Concentration. The x-axis depicts Hb concentration distributed into 2 g/dl bins from 4 g/dl to 20 g/dl. The y-axis shows the number of subjects in each bin. The image data were downloaded from the phone each day and stored on a computer. All data were transferred into MATLAB (Mathworks Inc, Natick MA.) for analysis.

In some implementations, a standard iPhone can be used to take the images of the conjunctivae of the patients. Additionally, Hb of the patients was measured via blood test. The iPhone can allow for images to be stored as RAW images in a portable network graphic (PNG) file format. The RAW images can provide data directly from the camera sensor without the typical processing and compression that occurs with typical images such as Joint Photographic Expert Group (JPEG). The PNG file of a RAW image can include metadata associated with the image (e.g., indicative of time, location, camera settings, etc.).

RAW images can contain detailed information of the captured image. But the RAW images may not be in a format that can be easily displayed. In the above-mentioned study, the RAW image file is manipulated to obtain accurate color information.

Each processed image can be stored in a custom JRI file format (James Rayner Image file) which can also contain all the metadata from the original RAW image file (e.g., PNG file). Each JRI file can contain the raw image stored as a 4032 X 3024 X 3 matrix (e.g., matrix in MATLAB). This format can be similar to a RGB image format where each pixel in the image has a red, a green, and a blue value to describe its color. In RGB images each color channel can have one of 256 values which can be usually encoded in 8 bits. This can generate approximately 16.8 billion colors.

In some implementations, the received data characterizing the plurality of images is manipulated. For example, the received data characterizing the plurality of images can be unprocessed sensor data (e.g., in RAW photo format). The unprocessed sensor data can be processed by applying one or more of linearization, white balancing, demosaicing, color space correction and brightness contrast control. Manipulation of the unprocessed data can result in improvement of the color resolution (e.g., by using 32 bits to store the various color RGB color components) of images from the manipulated data compared to images from the unprocessed sensor data.

In some implementations, the unprocessed sensor data can be linearized or otherwise transformed (e.g., based on a look-up table associated with the sensor that generated the unprocessed sensor data). Transformation can include mapping each data value in the unprocessed data based on the look-up table. In some implementations, the color components of the transformed data (e.g., obtained by linearizing the unprocessed sensor data) can be adjusted (e.g., white balancing). This can include, multiplying the values associated with the various colors (e.g., red, blue, green, etc.) with various predetermined values. For example, values of data representative of red color in the unprocessed data can be multiplied by a red multiplier, values of data representative of blue color in the unprocessed data can be multiplied by a blue multiplier, values of data representative of green color in the unprocessed data can be multiplied by a green multiplier, etc. In some implementations, a demosaicing algorithm can be applied to the color adjusted data (e.g., obtained from white balancing). This can generate a 3-layered RGB data. In some implementations, a color space conversion algorithm can be applied to the output of the demosaicing algorithm. For example, the values of data associated with different colors of a given pixel (e.g., associated with a pixel of the sensor that generated the unprocessed sensor data) can be transformed (e.g., by multiplying a vector comprising the color values of the given pixel with a predetermined matrix). In some implementations, the color space conversion algorithm can generate 16-bit RGB image data. The brightness associated with this 16-bit RGB data can be adjusted. For example, the values in the RGB image data can be scaled (e.g., by adding a constant), applying a non-linear transformation or both.

For example, using the raw data each color level can be encoded with 32 bits giving 2³² or 4.3 trillion values for each channel. This can generate 2⁹⁶ or 8X10²⁸ colors. Each image from the Raw Image Directory can be converted by the above-mentioned technique which can be converted by MATLAB to a JRI file and stored in the Program Data Directory. A directory can call the RAW Image Directory created to store all the raw images gathered for further analysis.

In some implementations, images are analyzed using MATLAB. Each raw image file from iPhone is stored in a DNG format file. The raw file is processed using standard techniques to create a MATLAB RGB color format image file. The raw file can allow for custom processing to allow 32 bit (2³²) levels allowing very high color definition. In some implementations, RGB data can be obtained from preprocessed JPEG image / unprocessed RAW images by utilizing a 32 bit data per color channel (e.g., 32 bit channel for red, green, blue, etc.). This can increase color depth by many orders of magnitude and can provide other metadata to include in the processing (such as camera characteristics, entropy etc.)

A database is created to collate and organize raw images and other collected patient data in an anonymous format for later analysis. In one implementation, the database was created in MATLAB using a proprietary application that can scan all images in the program image database (PID) and analyze any images which are not already in the database. Each new image can be displayed for the user for visual inspection. The application can provide a graphical user interface (GUI) to allow the user to perform the following tasks (e.g., in a display space).

Some images in the database may not be suitable for processing. For example, images in which no image of the conjunctivae is present or the image is completely out of focus. Such images can be marked as INVALID. They remain in the database for completeness but will be ignored for further analysis.

Returning to FIG. 1, at 104, data indicative of a user selection of a first pixel of a first image of the plurality of images is received. The first image can be presented on a graphical user interface display space. In one implementation, a MATLAB application can be used by the user to select and zoom into a given image to select the region of interest (ROI) and pick a single pixel in the image of palpebral conjunctiva.

At 106, determining, a region of interest associated with the selected pixel is determined. In some implementation, the selected pixel is used as a “seed point” or a reference point. Based on the properties of the reference point, one or more neighboring pixels can be selected. For example, the determining of the region of interest can include selecting a plurality of pixels adjacent to the first pixel having a color parameter value within a predetermined range of a color parameter value of the first pixel.

In some implementations, an algorithm executed on the processor can select a test area of the conjunctiva by choosing nearby pixels with similar color values using a custom crystallization paradigm where the seed point serves as a starting point and adjacent pixels within certain specifications are added expanding outward from the seed point until an edge is detected, creating a crystal-like matrix of pixels. This selected region is stored in a patient database along with patient information including measured Hb.

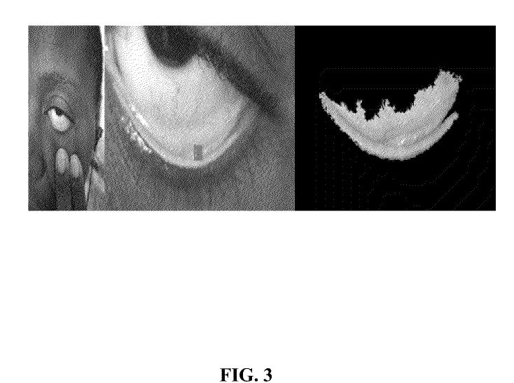

FIG. 3 illustrates an exemplary selection of a conjunctiva region. During this selection process images that are invalid, for example patient who has eyes closed, are excluded. The photograph on the left in FIG. 3 is the patient subject whose image was collected using the iPhone. The middle photograph is the subject’s eye in the MATLAB application for selection of conjunctiva region to be used in analysis. The gray square is the selected pixel representing best conjunctiva color. The photograph on the right in FIG. 3 shows the region of the patient’s palpebral conjunctiva that was selected by the algorithm in the MATLAB application. In some implementations, if an image is not invalid, the user is prompted to select a pixel in the center of the conjunctive with a mouse click or using their finger to tap on a screen.

The algorithm can select a region of interest (ROI) as a rectangle of standard size around the selected point. The coordinates of the corners of the rectangle are added to the database. This can allow for analysis of the ROI instead of the entire image. The pixel selected by the user can be saved as a seed or reference point.

The results of this processing yield the following for valid images.

In some implementations, due to memory / processing speed constraints, large image files are not stored in the database. After the image is analyzed as described above, the ROI coordinates, seed point coordinates, and a binary mask for quick selection of pixels from the ROI is stored in the Patient Image Database.

Selecting a region of the conjunctiva for analysis by hand can lead to variable results as different users may pick different regions for analysis. Selection of a region can be improved (e.g., for repeatability and accuracy) by automating the selection process based on predetermined selection criteria. The predetermined selection criteria can be based on the properties of the seed pixel or the image of the conjunctival region. For example, the conjunctival region is naturally bounded above by the white sclera and below by the lower lid margin. This property can be used to determine predetermined selection criteria for automatic extraction of pixels in the ROI.

The ROI can be converted from the RGB color space to the LAB color space as it can more closely align with how humans perceive color. The LAB color space (also known as CIELAB) can be more perceptually uniform. It can include three components: “L” (lightness), “A” (e.g., red/green component), “B” (e.g., blue/yellow component). In some cases, RGB may not correspond well with how humans see and may be better for image display. LAB can provide a smoother color space for searching. A difference between the color of the seed point (e.g., represented by a color value) and the color of all pixels in the ROI (e.g., a plurality of color values) can be calculated. Then, the square of the difference can be calculated to highlight variation and to make all differences positive. Using this map of differences, all pixels close in color and connected to the original seed point can be selected (e.g., using the MATLAB “imsegfmm” function). This can provide a robust method of quickly selecting pixels automatically as shown above.

Returning to FIG. 1, at 108, a first plurality of parameters associated with the region of interest is determined. In some implementations, a matrix comprising a first plurality of rows and a second plurality of columns are generated. The first plurality of rows are representative of the plurality of images and the second plurality of column are representative of the first plurality of parameters.

In some implementations, the plurality of parameters can be calculated (e.g., using MATLAB) by analyzing the selected region. These parameters can represent information such as average brightness of the image, average value of red image component, camera information such as whether flash was used, and entropy of the image. The parameter values can be included in a row in the matrix. In other words, each column represents the values of a given parameter for each image. The final column is the measurement Hb via CBC. Each row can be representative of an image.

In some implementations, the above-mentioned matrix can be used to perform a linear regression (or other correlation models including machine learning algorithms or neural network type processes) and create a predictive model for analytes in blood (e.g., Hb, bilirubin, etc.) using images in the database (e.g., images of the right eye). For example, stepwise linear regression can receive as an input a group of prediction parameters (described below) and known results and calculate the best model (e.g., linear regression model) to fit the data using a subset of the parameters.

In some implementations, data from the imaging of the contralateral eye (e.g., left eye) was then analyzed by the algorithm resulting in predicted IHB values. In some implementations, stepwise linear regression can be used to create a model which can predict IHB from a number of parameters. The stepwise regression can automatically remove terms with poor correlation to IHB. Hue is a component of the HSV color space. It can range between 0 and 1. Hue can be indicative of color component with pure red at 0 transitioning to green at 0.33 then to blue at 0.66 and back to red at 1. Entropy can be a measure of the spatial complexity [Yi-Ming Chem et al. Examining palpebral conjunctiva for anemia assessment with image processing methods, https://pubmed.ncbi.nlm.nih.gov/28110719/]. A single homogenous color can have low entropy and a complicated pattern, such as conjunctival blood vessel patterns, can have a high entropy.

In some implementations, a mobile device (e.g., smartphone) can obtain and process digital images of the palpebral conjunctiva to predict Hb concentration.

As described above, some implementations of the current subject matter can include an interactive application that can allow a user to take pictures of the conjunctiva (e.g., inner layer of eyelids) and execute an image analysis technique (e.g., an image analysis algorithm executed on the smartphone). The image analysis algorithm can process the raw images captured by the camera to improve the quality of the captured image presented to the user. For example, the RAW images can provide data directly from the camera sensor without the typical processing and compression that occurs with typical images. The image analysis algorithm can be processed in real-time to generate a processed image (based on processed RAW image data). For example, the unprocessed sensor data can be linearized or otherwise transformed (e.g., based on a look-up table associated with the sensor that generated the unprocessed sensor data). Transformation can include mapping each data value in the unprocessed data based on the look-up table. The image analysis algorithm can account for white balance, ambient lighting, glare, pigmentation of the surrounding skin, and detect borders of conjunctiva separated by other anatomical features in the image such as the sclera (white), pupil (black), edges of the eyelid, and the like. Processing of the raw image can enable improved detection of Hb.

Unlike some approaches where spectroscopic behavior is modeled, some implementations utilize the images directly (e.g., the raw images or unprocessed sensor data), which can enable faster and more accurate detection of Hb. For example, some implementations of the current subject matter do not utilize spectral-super resolution spectroscopy, which mathematically reconstructs hyperspectral data from RGB data. As another example, some implementations of the current subject matter does not determine or solve for multiple wavelengths within the visible light band that were not acquired by the sensor (e.g., that are not RGB values). Determining or solving for multiple wavelengths within the visible light band that were not acquired by the sensor (e.g., that are not RGB values), can be mathematically complex and require significant compute resources, which may be unsuitable for a mobile device (e.g., mobile phone, tablets, and the like).

In some implementations, raw sensor data can be used from the camera array to create color spaces that can be in the format of RGB, hue, saturation, lightness (HSV), or LAB color formats. This is more direct than using RGB data and attempting to recapitulate spectroscopic data through a sophisticated super-resolution spectroscopy (SSR) model. In some implementations, by accessing the sensor data directly, floating point numerical methods that can define a color space with very high resolution can be used. Thus unhindered by inaccuracies introduced by image formatting or compression. This approach can improve the speed of programming and can create efficiencies that occur prior to the look up table/predictive model part of the overall Hb prediction algorithm. Those efficiencies can result from the lack of image de-compression in the RGB space. The starting spectral data can be the actual raw data (outside of any pre-established standardized color space) acquired from the camera sensor itself.

### Example 1

In one example, conjunctiva images and laboratory data (e.g., within 4 hours of image capture) were collected from 142 patients. Computer algorithms were developed to process image related data to correlate with laboratory derived Hb concentrations with the goals of developing an in-phone application to obtain and process imaging data and determine Hb rapidly, in real-time and non-invasively solely using the handheld smartphone. A stepwise linear regression utilizing variables derived from the RAW digital image was constructed and trained to maximize correlation to individual CBC values from the right eye of 142 patients. This regression model was then used to predict Hb concentration from images obtained the left eye from the same 142 patients. In some implementations, performance of the statistical association can be enhanced by obtaining a true Hb value for a user (which can be routine in clinical practice) which will serve to calibrate and anchor the stepwise linear regression which will serve to further improve accuracy. In some implementations, as described above, a look-up table approach can be used to estimate Hb values from the algorithm performance.

Statistical Methods will now be described. CBC HB versus IHB (e.g., with flash or without flash) can be determined for both the left eye and the right eye of a given patient. For example, images from the right and the left eyes of the same patient (with identical gold standard Hb as measured from their blood by a hospital lab CBC test) are used as comparisons. A paradigm (e.g., linear correlation model) can be used to model CBC HB (e.g., gold standard, measured) based on IHB (predicted, calculated based on image) (e.g., of the right eye and/or the left eye). The model can allow for prediction of HB based on images of the left and/or right eye. A three-way interaction term is included in the linear model. The linear model can generate a correlation curve (e.g., for left eye, right eye, with / without flash). In some implementations, differences in slope and intercept of correlation plots generated by the model indicate that flash may not be desirable.

Error of IHB to CBC HB defined as difference between blood measured CBC and IHB is modeled (e.g., by flash and side). A two-way interaction is included to allow for differences in slope and intercept by each level of flash and side.

Right and Left eye HB measurements are averaged for subsequent analysis (e.g., average between right and left Image HB). Error of right and left averaged IHB and CBC HB (e.g., difference between blood measured CBC HB and right and left averaged IHB) can be modeled by flash.

Blood determined HB versus right and left averaged Image HB will now be described. Right and left averaged Image HB with no flash is analyzed. Bland-Altman plots (e.g., difference between blood measured CBC HB and right and left averaged IHB vs the mean of blood measured HB and right and left averaged IHB) are used to further assess the right and left averaged IHB measure for bias and precision relative to the CBC HB measure.

Error (e.g., difference between blood measured CBC HB and right and left averaged Image HB) was modeled by averaging HB (e.g., (blood measured CBC HB + right and left averaged Image HB)/2) to understand underlying trends with error and increasing average HB level.

Clinical usefulness of eye determined HB will now be described. To test clinical usefulness of the right and left averaged eye determined HB measure, blood determined measures of HB and eye determined measures of HB is categorized as anemic (e.g., less than 12.5 for women, less than 13.5 for men) or not anemic. Agreement between blood and IHB measures is assessed using a generalized linear model for binary outcomes to model the proportion of patients who are anemic by blood test considering their eye determined anemic status. Models are evaluated for significance, sensitivity, specificity, and accuracy (AUC). The sensitivity, specificity, and AUC values for the models are reported as indicators of the usefulness of the model in predicting anemia (e.g., because p-values alone may not represent the strength of a prediction). This assessment is repeated for cutoffs of 7 and 9 (used to determine if transfusion of packed red blood cells (PRBC) are required, a clinically usefull parameter). To visualize the tradeoff of sensitivity/specificity with increasing right and left Image HB (ROC), the proportion of patients who are anemic by CBC are modeled by right eye and left eye IHB (continuous).

All models can be analyzed using proc glimmix. Nesting for patient repeated measures can be accounted for in all models. Family-wise error rate (alpha) is maintained at 0.05 using the Holm adjustment for multiple comparisons where appropriate/adjusted p-values are reported. Classic sandwich estimation is used to adjust for any model misspecification in the models. All statistical analyses are performed using SAS version 9.4 (The SAS Institute; Cary, NC).

Results will now be described. During an enrollment period, images from 142 unique patients 52% of whom were male were obtained. Hb concentration ranged from 4.7 to 19.6 g/dl. FIG. 2 illustrates the exemplary distribution of Hb concentration of patients. This distribution fits a normal distribution. The average patient age is 50 years and the patient age range is 19-93 years. All patients had pulse oximetry oxygen saturation in the normal range and for those patients who had serum bilirubin ordered (41% of enrolled patients), this value was not elevated.

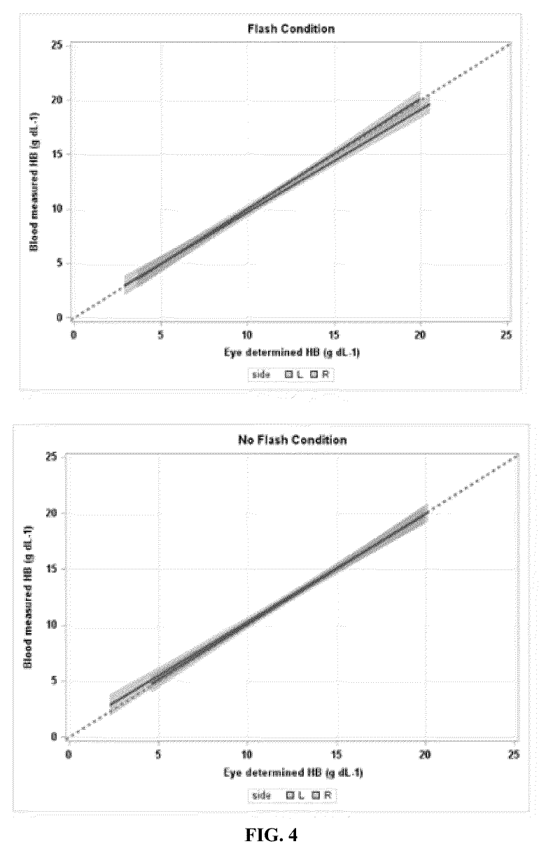

CBC HB versus IHB (with and without flash) for left eye and right eye of a given patient will now be described. FIG. 4 illustrates correlation between IHB and CBC HB (p<0.001) (with and without flash) for left eye and right eye of the given patient. These graphs illustrate the relationship of the HB obtained from a CBC to that of the predicted HB using this method for the conditions of flash and no flash for right eye images and left eye images. Blue and red shaded regions indicate 95% confidence intervals for slope. Dashed gray line represents difference between CBC HB and Image HB.

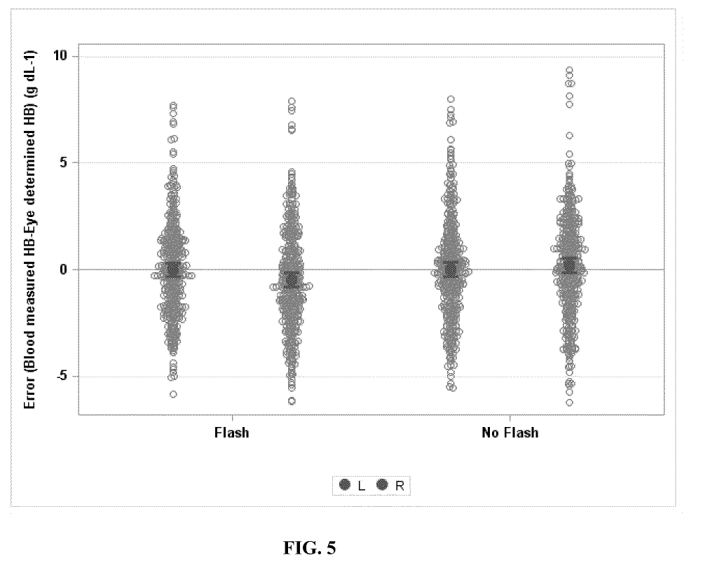

FIG. 5 illustrates that error for image HB relative to CBC HB is larger for right eye with flash (-0.48 [-0.86, -0.1] g dL-1) than left eye with flash, left eye without flash and right eye without flash (p=0.0053, p<0.0001, and p=0.0076). See Table 1. Blue and red dots represent estimated mean error for right and left eye with flash. Gray dots represent individual measures of error per patient. Error is not significantly different between the right eye and left eye for the no flash condition (p=0.5087). Error for eye averaged HB is larger for flash (-0.25 [-0.61, 0.1] g dL-1) than the no flash condition (0.1 [-0.26, 0.47] g dL-1, p=0.0001)

Table 1

Error (difference between CBC HB and IHB) by flash condition and side

| flash condition | side | Error [95% CI] g dL⁻¹ |
|---|---|---|
| Flash | L | -0.02 [-0.38, 0.35] |
| Flash | R | -0.48 [-0.86, -0.1] |
| No Flash | L | 0.02 [-0.37, 0.4] |
| No Flash | R | 0.18 [-0.21, 0.57] |

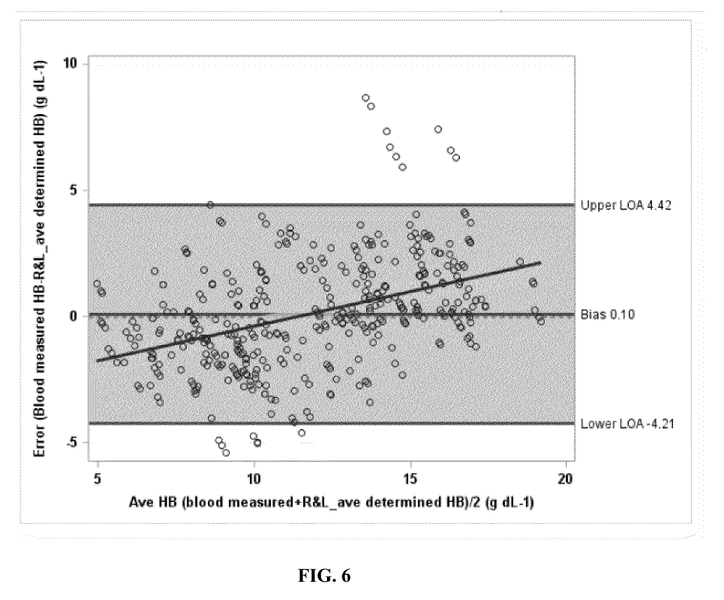

CBC HB versus right and left averaged IHB will now be described. FIG. 6 illustrates an exemplary Bland-Altman plot of right and left averaged IHB versus CBC HB. The plot indicates a bias of 0.10 and limits of agreement -4.21 to 4.42. Error increases with increasing average HB values (p<0.001). Right and left averaged IHB can overestimate HB compared to CBC HB in the lower range of HB (<11). Right and left averaged IHB can under estimate HB compared to CBC HB in the higher range of average HB (>11). The pink shaded area represents limits of agreement. Model fit for error by increasing average HB is represented by the solid blue line. Blue shaded region represents slope 95% confidence intervals and gray dotted line represents zero error. Knowledge of these biases could be used to adjust prediction model to offset error and obtain a better correlation.

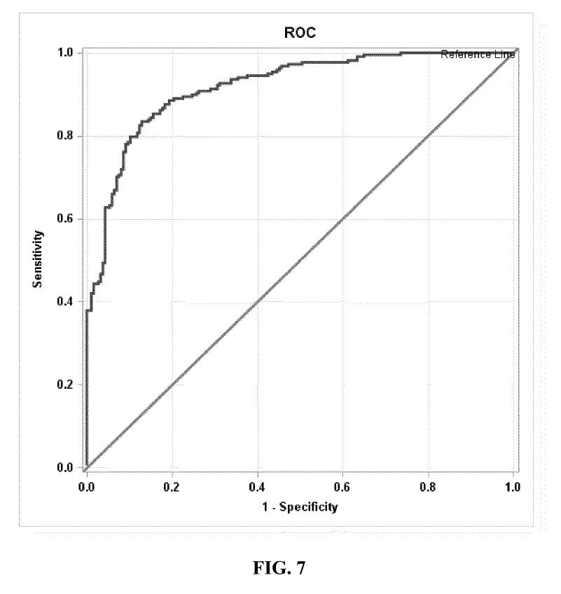

Clinical usefulness of image HB will now be described. Accuracy, sensitivity, and specificity of IHB for predicting anemia was 82.9 [79.3, 86.4], 90.7 [87, 94.4], and 73.3 [67.1, 79.5], respectively. Accuracy, sensitivity, specificity, false positive rate, and false negative rate for transfusion cuts offs are presented in Table 2. FIG. 7 is a graph illustrating the tradeoff of between sensitivity and specificity of a classical receiver operator curve. The curve indicates the change in sensitivity / specificity due to change in IHB (ROC). The curve can be used for predicting the proportion of patients who were anemic by CBC HB. The area under the curve (AUC) and the point on the curve (in red) which is closest to the right upper corner where sensitivity and specificity are maximal (1.0) can define the clinical usefulness of this method for predicting anemia using the WHO criteria.

Table 2

Clinical usefulness of Image HB

| Predicted Outcome | R&L averaged Image HB | Estimate [95% CI] |
|---|---|---|
| Anemia <12.5 Women <13.5 Men | Accuracy | 82.9 [79.3, 86.4] |
| | Sensitivity | 90.7 [87, 94.4] |
| | Specificity | 73.3 [67.1, 79.5] |
| | False Positive Rate | 26.7 [20.5, 32.9] |
| | False Negative Rate | 9.3 [5.6, 13] |
| Transfusion Low <7 | Accuracy | 91.1 [88.3, 93.9] |
| | Sensitivity | 40 [25.7, 54.3] |
| | Specificity | 97.5 [95.9, 99.1] |
| | False Positive Rate | 2.5 [0.9, 4.1] |
| | False Negative Rate | 60 [45.7, 74.3] |
| Transfusion high <9 | Accuracy | 81.4 [77.6, 85.2] |
| | Sensitivity | 51.2 [42.4, 60.1] |
| | Specificity | 94.6 [92, 97.3] |
| | False Positive Rate | 5.4 [2.7, 8] |
| | False Negative Rate | 48.8 [40, 57.6] |

In one implementation, the database can include the following information: Image filename; patient number (each patient has unique number for identification); Hb measured by blood test; logical value: 1 or 0 for valid and invalid image, respectively; region of Interest Coordinates; selected Point by User (specified in full image and ROI coordinates); binary mask for quick selection of pixels of interest from ROI.

The database can allow for quick access any valid image and extract a high color resolution image of the pixels of interest in the conductive from that image. A prediction parameter (extracted from the image) is a value which encapsulates information in the image. For example, it can be the average red value of all selected pixels, camera information form the image metadata, etc. The prediction parameters can be used in mathematical model to predict Hb from the image data. Desirable parameters can be determined by testing the predictions against measured Hb levels.

In some implementations, the following parameters can be generated:
BRIGHT - Average value of gray scale image
R0 - Average value of red component of all pixels
R1 - Average value of red component of pixels between 2nd and 12th percentiles
R2 - Average value of red component of pixels between 50th and 52nd percentiles
R3 - Average value of red component of pixels between 88th and 98th percentiles
G0 - Average value of green component of all pixels
G1 - Average value of green component of pixels between 2nd and 12th percentiles
G2 - Average value of green component of pixels between 50th and 52nd percentiles
G3 - Average value of green component of pixels between 88th and 98th percentiles
B0 - Average value of blue component of all pixels
B1 - Average value of blue component of pixels between 2nd and 12th percentiles
B2 - Average value of blue component of pixels between 50th and 52nd percentiles
B3 - Average value of blue component of pixels between 88th and 98th percentiles
RPVM - Average value of red component of pixels between 40th and 60th percentiles
GPVM - Average value of green component of pixels between 40th and 60th percentiles
BPVM - Average value of blue component of pixels between 40th and 60th percentiles
ENTROPY - entropy of grayscale image
HHR - high hue ratio
H - average value of hue from HSV colormap
L - average value of lightness from LAB colormap
FLASH - Did camera flash fire for this image?
BE - Camera metadata: Baseline Exposure
ASN1 - Camera metadata: AsShotNeutral1
ASN2 - Camera metadata: AsShotNeutral2
BV - Camera metadata: BrightnessValue
Hb - Hemoglobin measured. Not used for predictions rather reference for testing.

### Example 2

In some implementations, the image analysis algorithm can process images from a smartphone-based digital camera for estimating Hb concentration that can maximize color resolution. One implementation of the image analysis algorithm has been developed and tested respectively in a prediction and validation model separately in two separate cohorts of emergency department (ED) patients. Initially, images were collected from 142 patients using a smartphone camera and determined image characteristics which are useful in constructing a correlation between photographs of the conjunctiva and the actual laboratory-determined Hb concentration value in an ED derivation patient cohort. Then, this correlation was implemented to estimate Hb concentration and predict anemia in 202 new ED patients, in addition to the derivation cohort. Identifying those patients who met WHO defined anemia limits and transfusion thresholds. This required using the final validation data set of 344 patients in a predictive model using k-folds testing iteration on a randomly selected 10% of the total population ten times.

Images were collected from patients who were in the emergency department (ED) at Rhode Island Hospital between Dec. 1, 2018 and Aug. 31, 2019. Inclusion criteria included having a complete blood count (CBC) obtained as part of the patients' care within 4 hours of digital image acquisition, ability to provide informed consent and ability to expose the palpebral conjunctiva of both eyes. Patients with injury or infection of the eye were excluded. Patients could be lying supine or in a sitting position and were asked to remain still and pull down their lower eyelid to expose their conjunctiva. Images were obtained under ambient indoor light. For the initial algorithm derivation phase (phase 1) of the study, 32 images were obtained from each patient. Eight images of the conjunctiva with a standard color reference (colorchecker, Passport Photo, x-rite; xritephoto.com) adjacent to each eye with the conjunctiva exposed were recorded in raw and JPEG format with and without built-in flash. The remaining 24 images were obtained without the color reference as close to the conjunctiva as could be clearly focused. Care was taken to minimize glare from ambient light sources and minimize movement. Images were obtained using Halide (Chroma Noir LLC San Francisco, CA.) application on an iPhone 7 Plus (Apple Inc, Cupertino, CA.). Demographic information including gender, age, vital signs and Massey skin color rating [Park SM, Visbal-Onufrak MA, Haque MM, Were MC, Naanyu V, Hasan MK, et al. mHealth spectroscopy of blood Hb with spectral super-resolution. Optica. 2020] was collected on a data collection form along with hospital laboratory reported lab tests including Hb, total and direct bilirubin. time of collection. To reduce variability, all imaging data were collected by a single operator who developed the data collection methods. Image data were downloaded from the smart phone each day and stored on a computer. All data were transferred into MATLAB (Mathworks Inc, Natick MA.) for analysis.

In the validation phase of the study (phase 2) a new cohort of 202 ED patients were imaged while the data collection operator was unblinded to patient Hb values. For these patients, 3 images of each eye, total 6 per patient, were obtained without flash in RAW mode only. All other methodology remained the same in phase 1 of the study.

Demographic and laboratory data were obtained from the patient’s electronic medical record. Massey score (1-9: 1 being light skin and 9 being dark skin) was determined visually by a single observer using a standard skin tone chart and recorded in the database [Park SM, Visbal-Onufrak MA, Haque MM, Were MC, Naanyu V, Hasan MK, et al. mHealth spectroscopy of blood hemoglobin with spectral super-resolution. Optica. 2020].

Images were analyzed using MATLAB. Each raw image file from the iPhone was initially stored in a Portable Network Graphics (PNG) format file. Raw images provide data directly from the camera sensor without the typical processing and compression that occurs with common formats such as Joint Photographic Experts Group (JPEG). In each PNG file there is also a significant amount of metadata regarding time, location and camera settings.

The raw file was processed using standard techniques to create a MATLAB Red Green Blue (RGB) color format image file. The raw file enabled custom processing to allow 232 levels of color definition. A directory, “RAW Image Directory” (RID), was created to store all the raw images gathered for further analysis.

A raw image processing algorithm which maximized the color resolution of images was developed (e.g., as described in Massey D, Martin JA. The NIS skin color scale. Off Popul Res Princet Univ. 2003). Each processed image was stored in a custom “JRI” file format which also included the metadata from the original raw image PNG file.

Each JRI file contains the raw image stored as a 4032x3024x3 MATLAB matrix. This format is similar to an RGB image where each pixel in the image has a red, a green, and a blue value to describe its color. In typical RGB images each color channel can have one of 256 values. The custom algorithm used in this analysis generated a higher color definition image. Using raw data, each color level was encoded with 32 bits giving 4.3 trillion values for each channel and 8x1028 colors allowing for highly accurate color analysis. Each image from the RID was converted to a JRI file and stored in a Program Data Directory (PDD).

With respect to database development and image selection, a database was generated, in order to merge and organize information from each raw image with patient data. Using MATLAB, an application was created which displayed each new image for the user to visually inspect and provided a user interface to eliminate images which are not suitable for processing such as images which partially omit the conjunctiva, has poor lighting or are completely out of focus. For all valid images, the user was prompted to select a point within the palpebral conjunctiva representing the best conjunctiva color, with a mouse click. A region of interest (ROI) representing best color was visually selected as a rectangle of standard size around a selected seed point pixel (as illustrated in FIG. 3).

With respect to region selection and pixel extraction, images extracted from the ROI was converted to color spaces which provide increased contrast and a software algorithm to select a test area of the conjunctiva, using the selected seed point pixel (SP), by choosing nearby pixels with similar color values in a crystallization paradigm using the high contrast boundaries of the white sclera above and the skin below (see FIG. 3). This selected region was then stored in the database along with patient information including measured Hb. All further analyses of images were conducted using this selected region.

With respect to parameter extraction, image based parameters, 26 in total, were extracted from the ROI (see para [0092]-[00118). These parameters represent information such as average brightness of the image, average value of red image component, flash status and entropy of the image. Each parameter set was designated as a row in a table in MATLAB with each column representing the values of a given parameter for each image. The final column in this matrix was populated with the actual laboratory measured hemoglobin (HBl).

Stepwise regression analyses were performed using this matrix to develop a predictive model for hemoglobin (HBc) using the Phase 1 derivation data set of 142 patients. Later in the Phase 2 validation data set of 344 patients the predictive model was improved using k-means clustering iteration on a randomly selected 10% of the total population ten times. Predictions were tested on 30% of these images selected randomly combined with the collected validation data set.

With respect to image quality, to quantify image quality three separate observers rated image quality on a 3-point Likert scale for image domains of focus, extent of conjunctiva exposure and lighting, for each image obtained from 344 patients. Scale dimensions were categorized as “good”, “fair” or “bad” for each domain and total points were calculated for each image from each of the three observers. Gwet’s AC1 inter-rater reliability coefficients [Sumner R. Processing RAW Images in MATLAB. Opt Commun. 2013] were calculated for images that were “good”, “fair” and “bad”.

With respect to statistical methods, the derivation data set (Phase 1) was used in a generalized linear model for log normal data used to correlate laboratory tested Hb (HBl) against conjunctiva estimated Hb (HBc) in flash use and eye laterality. A 3-way interaction term was included to allow differences in slope and intercept by each level of flash and side. Error of HBc to HBl (HBl-Hbc) (normal distribution) was then modeled by flash use and eye laterality. A two-way interaction was included to allow for differences in slope and intercept by each level of flash and eye laterality. Right and left laterality HBc measures were then averaged for subsequent analysis (RLave). Error of RLave HBc to HBl (HBl-RLave HBc) was then modeled by flash only. Preliminary interim analysis showed averaging the right and left side HBc and the no flash condition to best approximate HBl values and these were used subsequently.

HBl versus RLave HBc: For the remainder of analysis, only images without use of flash RLave HBc (referred to as HBc for the remainder of analysis) were used in the analysis. Bland-Altman plots (HBl-HBc vs. the mean of HBl and HBc) were used to further assess for bias and precision relative to the gold standard. Error (HBl-HBc) was modeled by average hemoglobin ((HBl+HBc)/2) to understand underlying trends with error and increasing average Hb concentration. Trends seen (slope and intercept) with the phase 1 derivation data were used to correct predictions for the phase 2 validation data set.

Clinical Usefulness of HBc: To test clinical usefulness of the HBc measure, HBl and HBc were categorized as anemic (<12.5 for women, <13.5 for men) or not anemic. Agreement between HBl and HBc measures was assessed using a generalized linear model for binary outcomes to model the proportion of patients who were anemic by laboratory standard HBl versus HBc. Models were evaluated for significance, sensitivity, specificity, and accuracy (Area Under the Curve-AUC). The sensitivity, specificity, and AUC values for the models were reported as indicators of the usefulness of the model in predicting anemia, since p-values alone may not represent the strength of a prediction. This assessment was repeated for blood transfusion Hb cutoffs of 7.0 and 9.0 g/dl. To visualize the tradeoff between sensitivity and specificity with increasing HBc (Receiver Operating Characteristic Curve-ROC), the proportion of patients who were anemic by HB1 were modeled by HBc.

A general linear model was used to model error of HBc to HB1 (HBl-Hbc) (normal distribution) by average HB ((HBl+HBc)/2) and image quality. A two-way interaction was included to allow for differences in slope and intercept by each level of image quality. Bland-Altman plots were also created for the image quality subgroups. This same analysis was repeated for Massey Score categorized as low (1-3), medium (4-6) and high (7-9).

All models were analyzed using proc glimmix unless otherwise stated. Nesting for patient and reviewer repeated measures was accounted for by modeling random effects with the residual statement (gee). Familywise error rate (alpha) was maintained at 0.05 using the Holm adjustment for multiple comparisons where appropriate (adjusted p-values are reported). Classic sandwich estimation was used to adjust for any model misspecification. All statistical analyses were performed using SAS version 9.4 (The SAS Institute; Cary, NC).

Observer Agreement: Gwet’s AC1 statistic (first order agreement coefficient) [Sumner R. Processing RAW Images in MATLAB. Opt Commun. 2013] was calculated to assess agreement between observers, adjusting for agreement by chance. This approach to inter-rater reliability addresses a problem often found in calculating observer agreement using Cohen’s Kappa (x) when skewed or biased (e.g., some raters always rate high or low) distributions of applied ratings results in the inter-rater reliability being substantially lower than the percent agreement among raters [Sumner R. Processing RAW Images in MATLAB. Opt Commun. 2013]. Values of Gwet’s AC1 inter-rater reliability can be interpreted on the same scale as the κ statistic, <0.40 = poor; 0.40-0.75 = good; > 0.75 = excellent (21) [Wongpakaran N, Wongpakaran T, Wedding D, Gwet KL. A comparison of Cohen’s Kappa and Gwet’s AC1 when calculating inter-rater reliability coefficients: A study conducted with personality disorder samples. BMC Med Res Methodol. 2013].

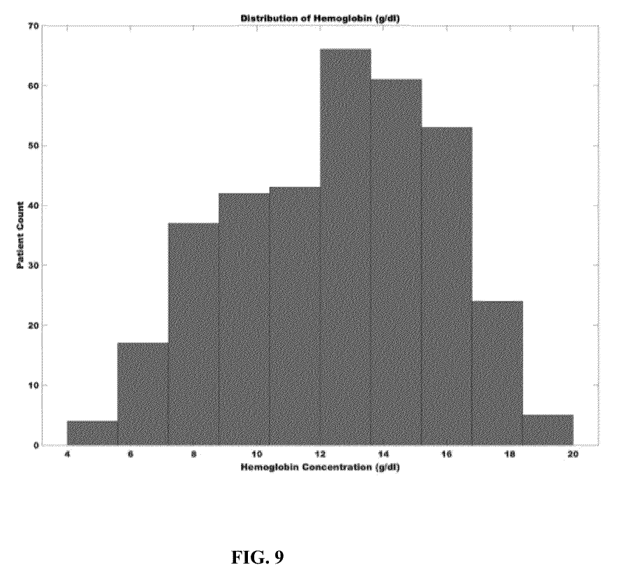

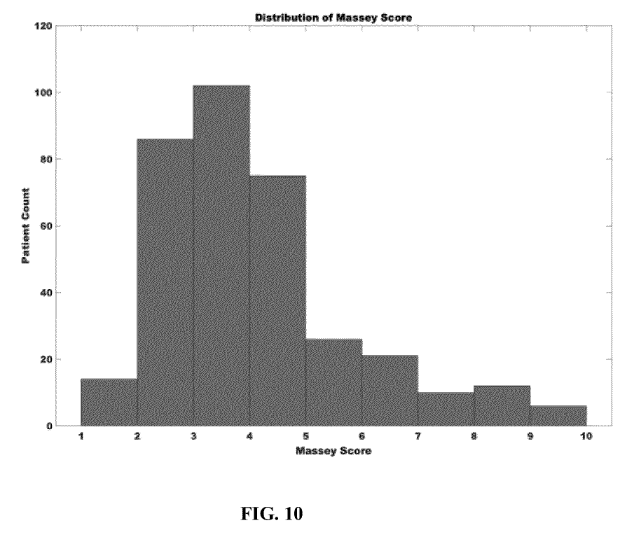

With respect to the results, during the two phases of enrollment, images from 344 unique patients 52% of whom were male were obtained. Hb concentration (mean 12.5) ranged from 4.7 to 19.6 g/dl. The distribution of Hb concentration is shown in FIG. 9 and fits a normal distribution. The average age was 53 years with a range of 19-96 years. All patients had pulse oximetry oxygen saturation in the normal range and for those patients who had serum bilirubin ordered (41% of enrolled patients), this value was not elevated. Mean Massey score (on a 1-9 scale) was 3.6 with a range of 1-9. The distributions of Hb concentration and Massey Score are depicted in FIG. 9 and FIG. 10, respectively. FIG. 10 illustrates a distribution of Massey Scores across 344 images of ED patients. The Massey Score is distributed into 9 bins from 1 to 9. Massey scores correspond to lighter skin and higher scores darker skin tone. While the Hb concentration was distributed normally, the Massey score was skewed toward lighter skin. The selection bias in skin color mirrored the emergency department population demographics.

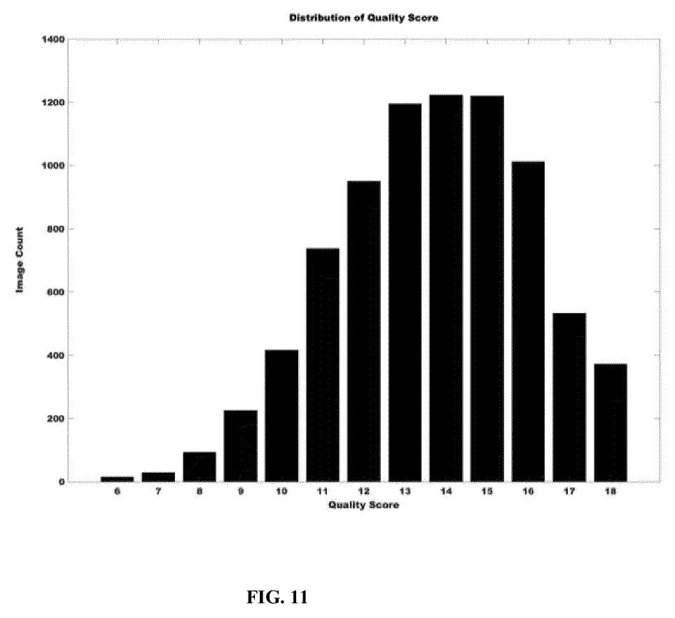

For phase 1, 1609 images of the conjunctiva from 140 individual patients were used in the analysis. For phase 2, 1722 images from 337 unique patients were used. One patient withdrew from the study after enrolling and 6 patients had invalid images. 5166 unique conjunctiva area ROI templates were generated by three observers from 1722 images corresponding to 337 unique patient Hb values. Each image was rated by three observers and a total image quality score was compiled for each image. The distribution of image quality scores is shown in FIG. 11. FIG. 11 illustrates Observer Image Quality score distribution. Higher scores correspond to better image quality comprised of the sum of focus, extent of conjunctival exposure, and lighting. Each domain was scored on a 3-point Likert scale and summed across three observers; the maximal score was 18.

There was good agreement between the three observers for images from participants with low (image quality score of 6-9; Gwet agreement coefficient 0.6-0.9) and high (image quality score of 16-18; Gwet agreement coefficient 0.6-1.0) quality. Agreement was poor for those which fell in the middle (image quality score 10-15; Gwet agreement coefficient 0.3-0.6).

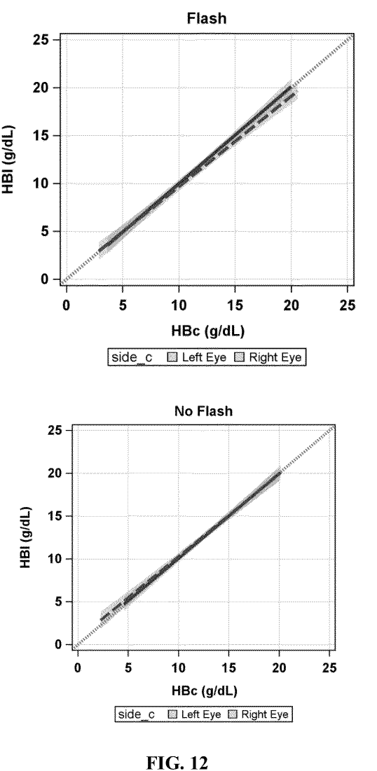

FIG. 12 illustrates correlation of HBc and HBl between images of the right and left eye in the Phase 1 derivation phase. The graph on the left shows the correlation when flash was used, and the graph on the right shows the correlation without flash. The x-axis depicts HBc in g/dl, the y-axis HBl. The red line corresponds to data from the right eye and the blue line from the left. The shaded areas depict the confidence intervals. The gray dotted line is the identity line (perfect agreement between HB1 and HBc).

HBc was significantly associated with HBl (p<0.001) in Phase 1 of the study. (FIG. 12). Error (HBl-HBc) for HBc was significantly larger when flash was used (-0.25 [-0.61, 0.1] g dL-1) (0.1 [-0.26, 0.47] g dL-1, p=0.0001), prompting elimination of flash use in phase 2 of the study. Error was not significantly different between the right eye and left eye images for the no flash condition (p=0.5087: 0.18 [-0.21, 0.57] versus 0.02 [-0.37, 0.4], respectively), so the average of both eyes was used for further analysis.

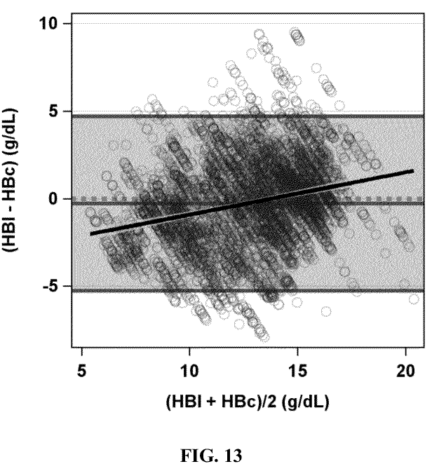

FIG. 13 illustrates a Bland-Altman plot for HBc compared to HB1 for all Phase 2 validation data. The average Hb concentration (HB1+HBc/2) in g/dl on the x-axis is plotted against Hb concentration error (HBl-HBc) in g/dl on the y-axis. The pink shaded area represents limits of agreement (Bias=-0.3, upper LOA=4.7, Lower LOA=-5.3). Model fit for error by increasing average Hb concentration is represented by the solid black line. Blue shaded region represents slope 95% confidence intervals. Green dotted line represents 0 error.

The Bland-Altman analysis shows a bias of -0.30 and limits of agreement -5.3 to 4.7 g/dl (FIG. 13) in the validation phase (Phase 2) of the study. Error was found to trend with increasing HB1 values (slope 0.27 [0.19, 0.36] and intercept -3.14 [-4.21, -2.07] (p<0.001). HBc tends to overestimate Hb compared to HB1 in the lower range of HB (<11 g/dl). HBc tends to underestimate Hb compared to HB1 in the higher range of average HB (>11 g/dl). This slope and intercept was used to correct for bias in the clinical results (Table 2) of the phase 2 validation study.. A 50% reduction was seen in the bias trend (slope and intercept) for phase 2 with this correction.

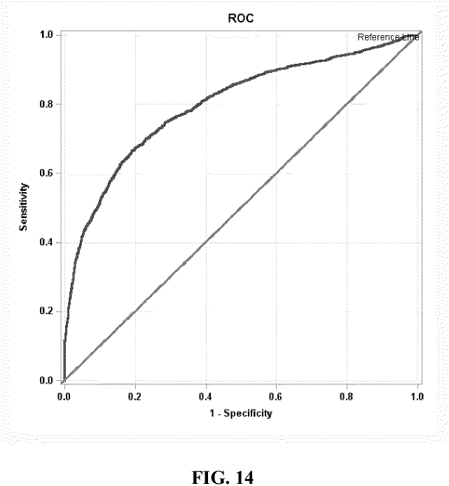

With respect to Phase 2: Clinical Results, accuracy, sensitivity, and specificity of HBc for predicting anemia was 72.6 [71.4, 73.8], 72.8 [71.0, 74.6], and 72.5 [70.8, 74.1], respectively. Accuracy, sensitivity, specificity, false positive rate, and false negative rate for Hb concentration transfusion thresholds are shown in Table 1. The tradeoff between sensitivity and specificity with increasing HBc (ROC), for predicting the proportion of patients who were anemic by HB1 is shown in FIG. 14. FIG. 14 illustrates Receiver Operator Characteristics of HBc using HB1 as the gold standard. The x-axis depicts 1- Specificity and the y-axis shows the sensitivity. The red line represents the ROC and the black line is the no-discrimination line. Area Under the Curve (AUC)= 0.8.

Table 3

Clinical Usefulness of Eye Determined HB

| Predicted Outcome | HBc predicting HBl | Estimate [95% Cl] |
|---|---|---|
| Anemia <12.5 Women <13.5 Men | Accuracy | 72.6 [71.4, 73.8] |
| | Sensitivity | 72.8 [71,74.6] |
| | Specificity | 72.5 [70.8, 74.1] |
| | False Positive Rate | 27.6 [25.9, 29.2] |
| | False Negative Rate | 27.2 [25.4, 29] |
| Transfusion Low <7 | Accuracy | 94.4 [93.7, 95] |
| | Sensitivity | 9.3 [5.9, 12.7] |
| | Specificity | 99.2 [99,99.5] |
| | False Positive Rate | 0.8 [0.6, 1.1] |
| | False Negative Rate | 90.7 [87.3, 94.1] |
| Transfusion high <9 | Accuracy | 86 [85, 86.9] |
| | Sensitivity | 39.6 [36.4, 42.7] |
| | Specificity | 96.1 [95.5, 96.7] |
| | False Positive Rate | 3.9 [3.3, 4.5] |
| | False Negative Rate | 60.4 [57.3, 63.6] |

Accuracy, sensitivity, specificity, false positive rate, and false negative rate for anemia as defined by the World Health Organization (WHO) and for transfusion thresholds are shown in this table. Hb values are in g/dl. Accuracy, sensitivity, specificity, false positive rate, and false negative rate are shown as predicted values [95% confidence intervals].

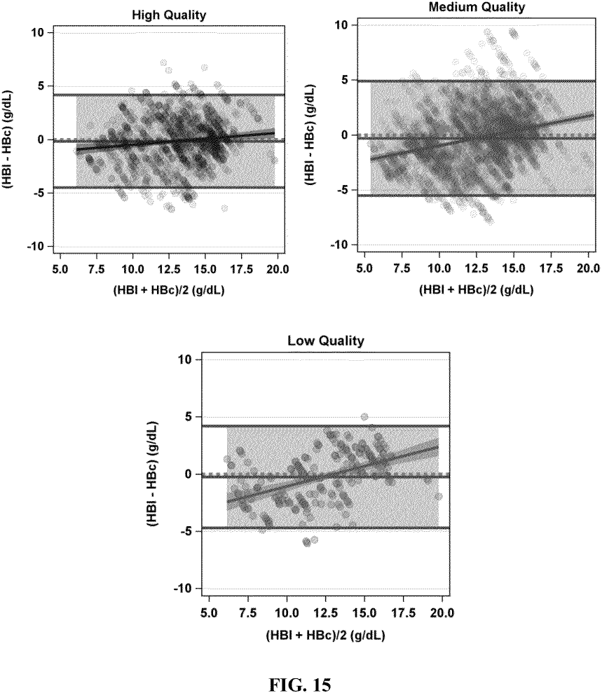

FIG. 15 illustrates a Bland-Altman plot for HBc compared to HB1. The average Hb concentration (HB1+HBc/2) in g/dl on the x-axis is plotted against Hb concentration error (HBl-HBc) in g/dl on the y-axis. The pink shaded area represents limits of agreement. Model fit for error by increasing average Hb concentration is represented by the solid lines. Shaded region represents slope 95% confidence intervals. Gray dotted line represents 0 error. The different colors represent data from different image quality ranges. Blue (a) represents high quality image data, red (b), medium quality and green (c), low quality colored lines show the respective slope and 95% confidence interval. When image quality was accounted for, error from images with high image quality scores had a smaller bias trend (slope) than medium or low quality images (0.11 [0.05, 0.18], 0.27 [0.22, 0.31], 0.35 [0.25, 0.46], respectively: both comparisons p=0.0001) and smaller limits of agreement ((-4.5, 4.2), (-5.5, 4.9), and (-4.7, 4.2)g/dl, respectively) (see FIG. 15).

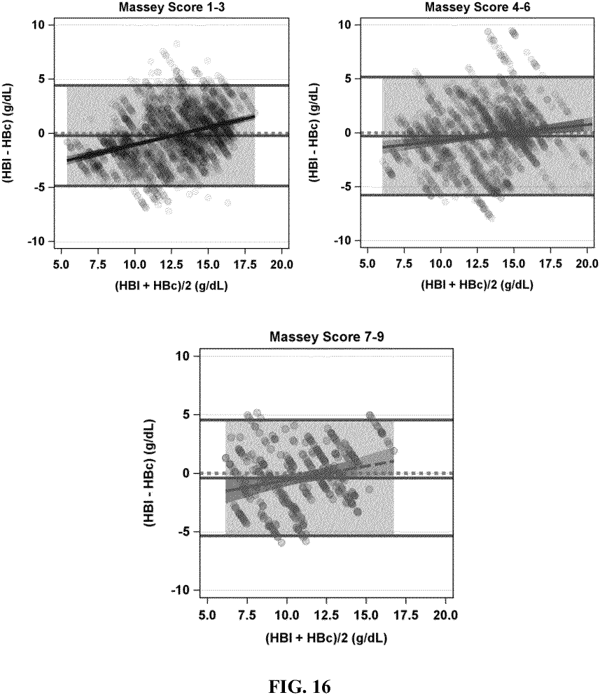

FIG. 16 illustrates a Bland-Altman plot for HBc compared to HB1. The average Hb concentration (HB1+HBc/2) in g/dl on the x-axis is plotted against Hb concentration error (HBl-HBc) in g/dl on the y-axis. The pink shaded area represents limits of agreement. Model fit for error by increasing average Hb concentration is represented by the solid colored lines. Shaded region represents slope 95% confidence intervals. Gray dotted line represents 0 error. Each color represents a Massey Score Grouping. Blue (a) representing Massey Scores of 1-3 (light skin), Red (b) 4-6 and Green (c) 7-9 (dark skin) colored lines show the respective slope and 95% confidence interval. When data were separated by Massey score grouping, there were no significant differences in limits of agreement, suggesting no effect of skin color on predicted Hb concentration (see FIG. 16).

Anemia, as classified by the World Health Organization, is among the greatest of health care concerns and a common comorbidity in both developed and developing countries. Noninvasive point-of-care testing devices for Hb have been studied to avoid venipuncture in determining Hb using standard methods. Point-of-care testing devices can serve as screens for anemia. Digitized imaging of the nail beds [ Fleiss JL. Measuring nominal scale agreement among many raters. Psychol Bull. 1971] and conjunctiva [ Saldivar-Espinoza B, Núñez-Fernández D, Porras-Barrientos F, Alva-Mantari A, Leslie LS, Zimic M. Portable System for the Prediction of Anemia Based on the Ocular Conjunctiva Using Artificial Intelligence. In: 33rd Conference on Neural Information Processing Systems (NeurIPS 2019). Vancouver, Canada; 2019] have reported acceptable accuracy for point-of-care testing devices which do not necessarily arise to a level of agreement that is +/- 1.0 g/dl. A cell phone enabled digital camera designed to acquire images of the conjunctiva would represent an advance over other methods in point-of-care testing since patients are already accustomed to recording “selfie” images that could be used in a medical application. This is an especially attractive opportunity for developing countries which may have sparse and rudimentary medical systems, however may be well-interconnected by established telecommunication networks.

This study was divided into two phases typical of look-up table algorithmic discovery: 1) Understanding the phenomenon under study; in this case the relationship between known Hb concentration and the spectral content of the conjunctivae using enhanced color discrimination data capture, and 2) Testing both the image capture and the nascent spectral-Hb relationship processing methods against a new population of research participants. The second phase of the study constitutes a real-world data collection set and shows that the level of accuracy was better than or showed equipoise with the Hb estimations provided by pulse co-oximetry [ Kalantri A, Karambelkar M, Joshi R, Kalantri S, Jajoo U. Accuracy and reliability of pallor for detecting anaemia: A hospital-based diagnostic accuracy study. PLoS One. 2010] . The pulse co-oximeter can be a more expensive and sophisticated device intended for operating room use for the ongoing intra-operative assessment of Hb. This technology may not have appeared in resource poor settings or in primary clinic practices thus far in significant numbers. As such, from a regulatory perspective, the pulse co-oximeter and its degree of accuracy may serve as a predicate device.

In the initial derivation phase 1 of the study it was shown that a predictive algorithm can be constructed using elements of a raw image obtained from a widely available smartphone. It was also shown that adjuncts such as color or white balance references and flash are not needed to construct this predictive model. It was also shown that there was a good correlation of Hb predicted by this approach to a laboratory confirmed Hb value from blood collected within 4 hours of the acquired photograph. Applying this algorithm to a larger patient cohort in phase 2 of the study validated the prediction of Hb using Bland-Altman analysis and showed that image quality was a determinant of prediction strength. It was also shown that the prediction was independent of skin color. These results set the stage for the development of an application within a smartphone which can not only acquire the image but also analyze the elements within the image to predict Hb concentration in real-time.

The present data shows that no flash digital photography was adequate to achieve the accuracies we report. We also determined that reviewers of the digital photographs while blinded to the Hb value were able to agree on poor and good appearing data. However, there was disagreement among the reviewers in regard to data that was borderline acceptable. The GWET AC inter-rater reliability analysis in lieu of the kappa statistic confirmed these results which is a common outcome in inter-rater agreement studies [ Sumner R. Processing RAW Images in MATLAB. Opt Commun. 2013].

Development of point-of- care digital health technologies has now been accelerated by the need to establish scalable telepresence medical systems in the COVID pandemic. A cell phone enabled medical application processing the algorithms described above can generate a Hb estimation with acceptable accuracy for point-of-care devices. This technology would fare well with other medical applications created to support remote medical services across a network. These networks must be ISO certified and HiTech and HIPAA compliant. Telecommunication systems can provide cybersecurity and interoperability in a cellular phone environment and thus enhance smart phone medical applications that utilize embedded sensors. This synergy is separate from traditional telemedicine platforms and offers patients a convenient and socially distant opportunity to access an important laboratory value. Interpretation of the Hb estimation would be the responsibility of the ordering physician; however, the opportunity to measure Hb remotely over a cellular network creates value, particularly for home care-based operations. The number of home care visits has increased significantly over the last several years [ McMurdy JW, Jay GD, Suner S, Trespalacios FM, Crawford GP. Diffuse reflectance spectra of the palpebral conjunctiva and its utility as a noninvasive indicator of total hemoglobin. J Biomed Opt. 2006]. Patient-centric approaches in health care may gain traction and patient interest beyond the immediate needs created by the global pandemic due to SARS-CoV-2.

A method to estimate Hb concentration using images of the conjunctiva obtained by a smart phone is described above. It is demonstrated, using data from patients in the ED, that estimation of Hb concentration by this method can be used as a screening tool for anemia and transfusion thresholds. Furthermore, it is shown that improvements in image quality and computational corrections can enhance estimates of Hb. There remains a significant step to package image collection, selection and computation into a self-contained application on a smart phone to create a point-of-care device which could be used both by clinicians in the hospital or office setting and by the lay public for screening or to enhance telemedicine encounters.

### Example 3

Anemia is a significant medical condition, which may lead to significant mortality and morbidity if undetected and left untreated [Nissenson, Allen R., Lawrence T. Goodnough, and Robert W. Dubois. “Anemia: Not Just an Innocent Bystander?” Archives of Internal Medicine 163, no. 12 (Jun. 23, 2003): 1400-1404. https://doi.org/10.1001/archinte.163.12.1400; Zakai NA, Katz R, Hirsch C, Shlipak MG, Chaves PH, Newman AB, et al. A prospective study of anemia status, hemoglobin concentration, and mortality in an elderly cohort: the Cardiovascular Health Study. Arch Intern Med., 165(19), 2214-20 (2005); Denny, Susan D., Maragatha N. Kuchibhatla, and Harvey Jay Cohen. “Impact of Anemia on Mortality, Cognition, and Function in Community-Dwelling Elderly.” The American Journal of Medicine 119, no. 4 (April 2006): 327-34. https://doi.org/10.1016/j.amjmed.2005.08.027. Culleton BF, Manns BJ, Zhang J, Tonelli M, Klarenbach S, Hemmelgam BR. Impact of anemia on hospitalization and mortality in older adults. Blood,107(10), 3841-6 (2006)]. Anemia is the deficiency in the concentration of healthy red blood cells circulating in the vascular system. The diminished oxygen carrying capacity from this deficit leads to tissue hypoxia and organ systems failure. Anemia is defined by the WHO as a hemoglobin (Hb) concentration below 12.5 g/dL for females and below 13.5 g/dL for males [World Health Organization (WHO), “Nutritional Anemias-Report of the WHO Scientific Group,” WHO Technical Report Series 415 (1968). Smith, Robert E. “The Clinical and Economic Burden of Anemia.” The American Journal of Managed Care 16 Suppl Issues (March 2010): S59-66]. According to the World Health Organization (WHO), anemia is the single largest global illness adversely affecting mortality and worker capacity, and The United States Department of Health & Human Services has declared anemia a significant public health concern. The National Anemia Action Council estimates that while 3.5 million people in the United States live with anemia, millions more remain undiagnosed. In developing countries where nutritional inadequacies and infectious disease, mostly parasitic infections such as malaria, are more prevalent, the effects of anemia are amplified, severely hindering children to reach their full genetically determined potential. The WHO estimates that 1.62 billion people worldwide suffer from anemia [de Benoist B et al., eds. Worldwide prevalence of anaemia 1993-2005. WHO Global Database on Anaemia Geneva, World Health Organization, 2008]. Anemia can influence physical function and work-force productivity through fatigue and weakness. Anemia also decreases myocardial function, leads to peripheral arterial vasodilation, and activates the sympathetic and renin-angiotensin-aldosterone systems. These effects influence the progression of diseases such as cardiac and renal failure [Toto, R. D., “Anemia of Chronic Disease: Past, Present and Future,” Kidney International 64, S20-S23 (2003); Pereira, A. A., and Sarnak, M. J., “Anemia as a Risk Factor for Cardiovascular Disease,” Kidney International 64, S32-S39 (2003); Silverberg, D. S., Iaina, A., Wezler, D., and Blum, M., “The Pathological Consequences of Anemia,” Clinical & Laboratory Haematology, 23, 1-6 (2001); Anand, I. S., Chandrashekhar, Y., Ferrari, R., Poolewilson, P. A., Harris, P. C., “Pathogenesis of Edema in Chronic Severe Anemia: Studies of Body-Water and Sodium, Renal-Function, Hemodynamic Variables, and Plasma Hormones,” British Heart Journal 70 (4), 357-362 (1993). Goldstein, D. Felzen, B., Youdim, M., “Experimental Iron Deficiency in Rats: Mechanical and Electrophysiological Alterations in the Cardiac Muscle,” Clinical Science (Colchester) 91, 233-239 (1996); Georgieva, Z., and Georgieva, M., “Compensatory and Adaptive Changes in Microcirculation and Left Ventricular Function of Patients with Chronic Iron-Deficiency Anemia,” Clinical Hemorheology and Microcirculation 17 (1), 21-30 (1997).]. Approximately the same number of people have anemia in the United States as have diabetes. In addition, anemia affects patients with a myriad of other diseases: at least 30% of all patients with cancer, an estimated 70% of all patients with HIV/AIDS, and 30-70% of all patients with rheumatoid arthritis [Weiss G, Goodnough LT. Anemia of chronic disease. N Engl J Med. 2005;352(10):1011-1023; Harrison L, Shasha D, Shiaova L, White C, Ramdeen B, Portenoy R. Prevalence of anemia in cancer patients undergoing radiation therapy. Semin Oncol 2001;28:54-9; Ludwig H, Fritz E, Leitgeb C, Pecherstorfer M, Samonigg H, Schuster J. Prediction of response to erythropoietin treatment in chronic anemia of cancer. Blood 1994;84: 1056-63; Rizzo JD, Lichtin AE, Woolf SH, et al. Use of epoetin in patients with cancer: evidence-based clinical practice guidelines of the American Society of Clinical Oncology and the American Society of Hematology. J Clin Oncol 2002;20:4083-107; Meidani, Mohsen, Farshid Rezaei, Mohammad Reza Maracy, Majid Avijgan, and Katayoun Tayeri. “Prevalence, Severity, and Related Factors of Anemia in HIV / AIDS Patients.” Journal of Research in Medical Sciences: The Official Journal of Isfahan University of Medical Sciences 17, no. 2 (February 2012): 138-42; Peeters, H R, M Jongen-Lavrencic, A N Raja, H S Ramdin, G Vreugdenhil, F C Breedveld, and A J Swaak. “Course and Characteristics of Anaemia in Patients with Rheumatoid Arthritis of Recent Onset.” Annals of the Rheumatic Diseases 55, no. 3 (March 1996): 162-68; Wilson, Alisa, Hsing-Ting Yu, Lawrence Tim Goodnough, and Allen R. Nissenson. “Prevalence and Outcomes of Anemia in Rheumatoid Arthritis: A Systematic Review of the Literature.” The American Journal of Medicine 116 Suppl 7A (Apr. 5, 2004): 50S-57S https://doi.org/10.1016/j.amjmed.2003.12.012.]. Patients with chronic kidney disease and those on chronic hemodialysis almost universally have anemia and often require treatment with expensive medications such as erythropoiesis-stimulating agents (ESA) and intravenous iron. Fatigue and weakness, although non-specific, are early signs of anemia which leads to diminished quality of life and loss of independence of many older adults, and has significant social and economic repercussions. The prevalence of anemia increases with age and on average affects about 13% of person’s older than 70 years of age [Salive, M. E., J. Cornoni-Huntley, J. M. Guralnik, C. L. Phillips, R. B. Wallace, A. M. Ostfeld, and H. J. Cohen. “Anemia and Hemoglobin Levels in Older Persons: Relationship with Age, Gender, and Health Status.” Journal of the American Geriatrics Society 40, no. 5 (May 1992): 489-96. https://doi.org/10.1111/j.1532-5415.1992.tb02017.x.]. The main causes of anemia in aging adults are other diseases (such as cancer and infectious diseases), or iron deficiency and malnutrition [Joosten, E., W. Pelemans, M. Hiele, J. Noyen, R. Verhaeghe, and M. A. Boogaerts. “Prevalence and Causes of Anaemia in a Geriatric Hospitalized Population.” Gerontology 38, no. 1-2 (1992): 111-17 https://doi.org/10.1159/000213315]. However, 1 in 5 elderly patients with anemia have no co-existing condition to account for a low Hgb concentration. Recent studies report that anemia in aging adults is an independent risk factor for decline in physical performance and is associated with higher mortality risks.

To screen for anemia, the physician can perform a visual inspection of the palpebral conjunctiva, send a blood sample for a CBC, spin a hematocrit or perform a bedside assay. There is also the copper sulphate test which is udes by blood banking organization to screen donors. The visual inspection of the conjunctiva by a physician is at best 70% accurate and is independent of the physician’s experience and training [Hung, O. L., Kwon, N. S.,Cole, A. E., Dacpano, G. R., Wu, T., Chiang, W. K., and Goldfrank, L. R., “Evaluation of the Physician’s Ability to Recognize the Presence or Absence of Anemia, Fever, and Jaundice,” Academic Emergency Medicine 7, 146-156 (2000)]. The CBC test is very accurate, but does not provide an immediate result. The CBC is invasive to the patient, associated with a significant cost, require laboratory resources and often not part of a routine physical exam. Other point-of-care devices including the pulse co-oximeter and Hemocue are inaccurate and invasive respectively, and introduce new costs to primary care practices. There is a need for a new device that can measure Hgb, non-invasively and inexpensively in real-time, with a high degree of accuracy and precision compared to the CBC. We envision a device that can be rapidly adopted by physicians, nurses and others in multi-tiered healthcare systems, and possibly even by patients, in a drive to advance self-care and create value in the systems that adopt it.

UNMET NEED: Busy front line medical providers including physicians, physician assistants and nurse practitioners are in need of a highly mobile point of care device that can immediately report a Hb value or at least determine a Hb value in the range where male and female adult patients are anemic. Knowing patients’ Hb value and whether they are anemic or not is a leading indicator used in the formation of diagnostic reasoning. Having that information up front as patients’ are interviewed would allow more expansive diagnostic possibilities and foster more patient shared discussions in lieu of the colloquial: “Let’s see what the lab results show”. In office settings, reliable, inexpensive and easy-to-use non-invasive bedside testing results would create value by driving the diagnostic process forward more quickly and may limit additional testing that would otherwise not be ordered if the practitioner knew reliably that patients’ are not anemic.

Within home care practice, there is a need for diagnostic devices that are portable while still maintaining the same standards of accuracy as traditional tests that one would receive in an office or hospital. Over the last two decades, there has been a resurgence in the prevalence of medical home visits, with the number of medical house calls rebounding from only about 984,000 in 1996 to 2.2 million house calls in 2016 and an additional 3.2 million visits made to assisted living facilities and group homes [Schuchman, Mattan, Mindy Fain, and Thomas Cornwell. “The Resurgence of Home-Based Primary Care Models in the United States.” Geriatrics 3, no. 3 (September 2018): 41. https://doi.org/10.3390/geriatrics3030041]. The driving factors behind this trend include a demographic shift toward a population with a larger elderly population, the desire for non-elderly consumers to avoid long wait times for primary care appointments and high costs associated with ED visits, and innovations that have made it possible to offer advanced medical technology within the home [Yasgur, Batya Swift. “Physician House Calls: An Old Model With a Modern-Day Twist.” Rheumatology Advisor, Apr. 29, 2019; https://www.rheumatologyadvisor. com/home/topics/practice-management/physician-house-calls-an-old-model-with-a-modem-day-twist/]. Hemacam can expand the ability of these providers to quickly detect Hb levels in these predominantly frail and elderly patients in a convenient manner without requiring additional equipment. Patients at risk for anemia could frequently monitor their Hb concentration, akin to home blood glucose measurements which are ubiquitous among patients with diabetes mellitus.

In addition, healthcare has long been based on the fee-for-service business model, but this has led to spiraling costs. The medical industry is now presciently moving to an outcome-based reimbursement strategy by focusing on device performance while controlling costs. This strategy may gain increasing importance over time and thus POC lab testing for Hb presents an opportunity that may create value in fast-paced and remote healthcare settings, and in home care practice.

A major trend in outpatient care is patient self-monitoring, which reduces cost and improves convenience for patients by minimizing the number of unnecessary appointments they must undertake for routine monitoring. This would improve quality of life particularly for hemodialysis patients, oncology patients, and patients recently discharged with significant anemia while still allowing clinicians to intervene early before patients experience life-threatening complications and minimizing. Crucially, a smartphone based mobile app is best positioned to accomplish this purpose because of its connectivity and already widespread ownership among Americans.

A hand held device that interprets a digital photograph of the conjunctivae can predict a patient’s Hb. The technology can be embedded in a mobile phone as a mobile health app. An analogy of the introduction of this technology can be found in the invention of the pulse oximeter; now ubiquitous in all phases of health care. During the early adoption of pulse oximetry, clinicians spoke in terms of “correlating” the Sp02 with a blood gas determined Sa02. Subsequent episodes in patient care when presenting a patient to another clinician included phraseology to the effect “the pulse co-oximetry correlated.....”. The Hemacam operating in a mobile medical application can be suitable for use in primary care settings, blood banking operations, and all phases of acute medical care. This includes pre-hospital care, walk-in medical centers, urgent care centers, emergency departments, and ICUs. In one implementation, 147 people have been studied with this app which constituted a training set.

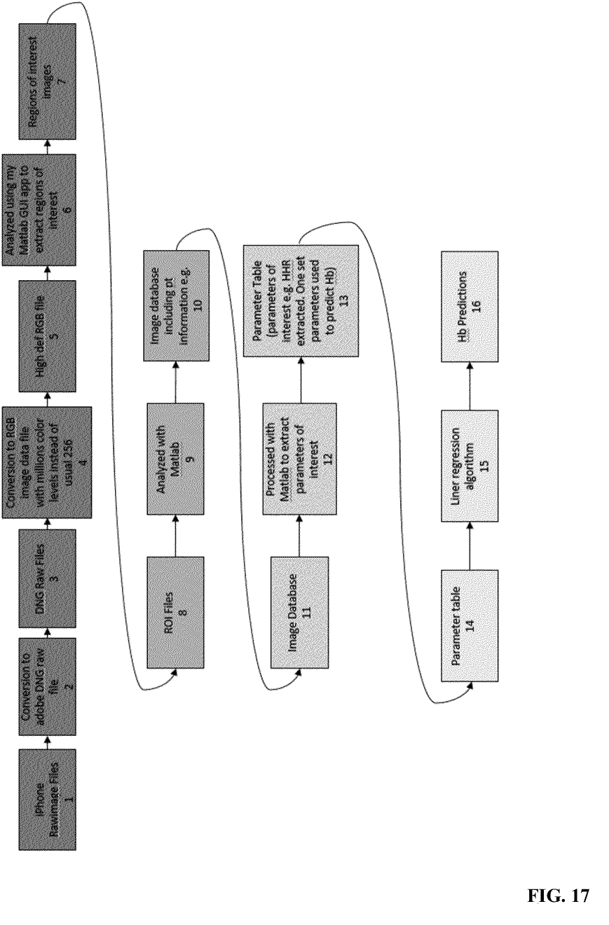

FIG. 17 illustrates an exemplary method of non-invasive measurement of Hb. This can be done, for example, using an application on a smart phone (e.g., an app on an iPhone).

With respect to the initial image processing (Steps 1-5), images taken with an iPhone camera may not be used for image processing. The images may be used for purposes such as documenting a trip or sharing an experience on social media. In these situations a visually appealing image of reasonable size allowing easy storage and transmission of the data may be desired. File formats such as j peg can produce compressed files to facilitate such activities but this can lead to a loss of information. While this is acceptable for most users’ purposes, it may not be desirable for applications described herein.

When predicting Hb levels by image processing one may not so interested in the appearance of the image but rather in the information contained within (e.g., within the color domain). Recently smartphone manufacturers have allowed users access to the inner workings of their cameras. In implementations described herein RAW image files generated by the iPhone camera may be used for analysis. RAW image files can contain the data that has come directly from the camera sensors with minimal processing and no loss of data.

In some implementations, images may not be processed with lossy compression like those used for sharing on social media. In some implementations, a processing system can make use of RAW images. These images may contain unprocessed data from the camera sensors.

RAW files can be converted to a more useful format. For example, a common format for images can be RGB where each pixel has a color specified by three numbers representing the amount of red, blue, and green in the color. RGB images can have 8 bits for each component meaning each color component can range between 0 and 255. The total number of possible colors is therefore 256x256x256 or roughly 16.8 million colors, which is more than the human eye can perceive. This system is ideal for viewing and sharing images online.

In some implementations, a high dynamic range (HDR) image can be obtained directly from the camera using the existing Bayer raw image table. This can be used in a Hemacam device (Computer Assisted Microscopy for Hematology, available from Fraunhofer Institute for Integrated Circuits IIS, Erlangen, Germany) as this data can be sourced directly from the image sensor in an iPhone. This can result in an improvement in the post-processed RGB images using a 32 bit processing feature for enhanced ‘human vision’ level of resolution. This can also result in increased speed of the calculations needed since post-processing is no longer needed. As a result, the raw image is obtained prior to any image processing. The Bayer table is a data representation of floating points for red, green and blue colors with an almost infinite resolution which can now be processed directly in the Hemacam algorithm. That algorithm remains an association between actual spectral response of the iPhone photo sensor to the conjunctival apparent redness and the patient’s true Hb value.

This direct method of sensor data interpretation can also be used with other matrix formats such as the Panchromatic cells, “EXR” color filter array, “X-Trans” filter, Quad Bayer and the Nonacell arrays. Each of these arrays differ by the ratio of green to red and blue sensors. In each case, for each of these array formats, a new derivation table would need to be created to associate spectral response of the iPhone photo sensor to the conjunctival apparent redness and the patient’s true Hb value.

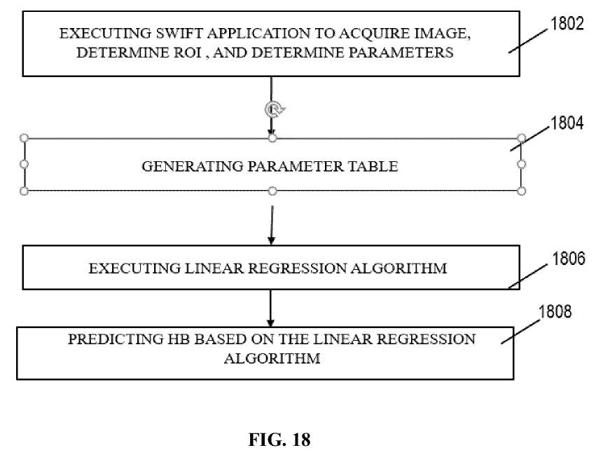

In some implementations, use of Bayer arrays can obviate the need for steps 1-10 in FIG. 17. Code written in Swift mobile app language can obviate the need for MatLab. For example, steps 9 to 14 in FIG. 17 can be implemented by Swift. The ROI selection may also conducted in Swift. Some implementations involve using DNG images produced by an iPhone camera to calculate a prediction for Hb level. This process involved converting Apple DNG files to a standard DNG format which can allow for uploading the images into Matlab. Using Matlab, the images are converted to RGB Matlab images for processing. The processing can allow for calculation of the parameters for linear regression algorithm in the implementation and hence Hb predictions. See FIG. 18.

Alternately, in some implementations, an iPhone application can receive the camera raw sensor data in Bayer RGGB format and processes it directly. Each pixel from the sensor can be converted to an RBG image represented in RBGAf format. This can allow for all possible information from the sensor to be captured for analysis as each pixel is now encoded with 16 bytes of data, with each color component now represented by a 4-byte floating point number. As illustrated in FIG. 18, the iPhone application can be based on Swift and SwiftUI and can allow the user to capture an image. The image is then processed directly (step 1802-1806), and a predicted Hb can be calculated (step 1808). This replaces steps 1-13 of FIG. 17. Matlab may be used offline for model refinement to allow updating of the model with new data.

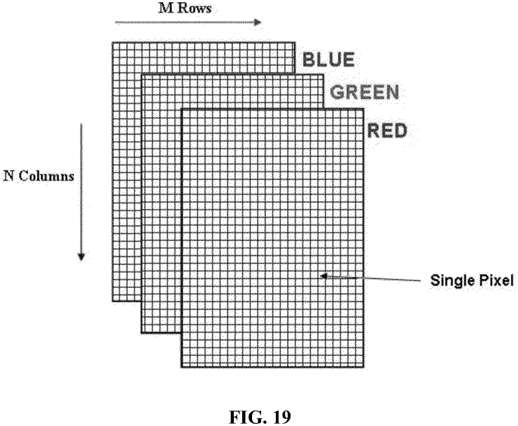

Higher color accuracy may be desirable for accurate estimation of Hb concentration. In some implementations, an N x M pixel RAW image can be converted to 3 N x M matrices where each matrix represents the red, green, and blue components of an RGB color image (see FIG. 19). Each component of the matrix can have 32 bits or approximately 4.3 trillion levels per component and a resulting 10²⁸ possible colors. This can allow for extreme sensitivity to color variations and leads to greatly increased accuracy of Hb predictions.

### Region of Interest Extraction (7-10)

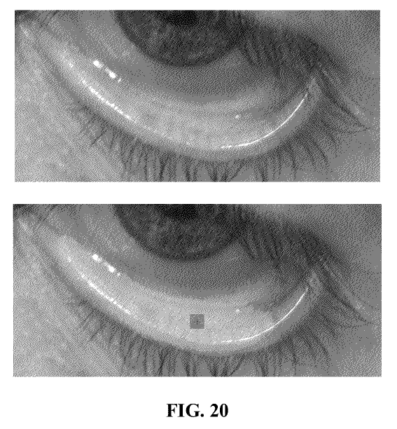

The image taken by the camera may include more than just the conjunctiva (e.g., portions of the eye adjacent to the conjunctiva). In some implementations, desirable portions of the image can be automatically extracted. Automatic region selection while good for usability may also leads to improved consistency of results as user error in region selection are avoided. In some implementations the correct region for processing can be extracted based on a user selected seed point, that is a selected point in the region of interest. Selecting one point may not be a great burden and in the app crosshairs on screen will help ensure the user is lined up with conjunctiva correctly. The crosshair intersection can then be used as seed point so region selection will be automatic. The algorithm uses the seed point and branches out to similar neighbors in a pseudo-crystallization paradigm to select the pixels of interest. FIG. 20 illustrates an exemplary selection of a seed point (red cross) on conjunctiva. The selection allows the software to extract desirable (e.g., ideal) region for analysis (shown in yellow).

In some implementations, homogeneity of human eyes, the typical colors as well as similar anatomy can be used to find the conjunctiva and select it from any image of an eye without any user input. This can allow any image to be used for analysis automatically without point selection and can make it possible to scrape suitable images from the internet and predict anemia in people in a completely automated fashion. In addition, such an automated system could autocorrect for lighting conditions by performing an automated white balance.

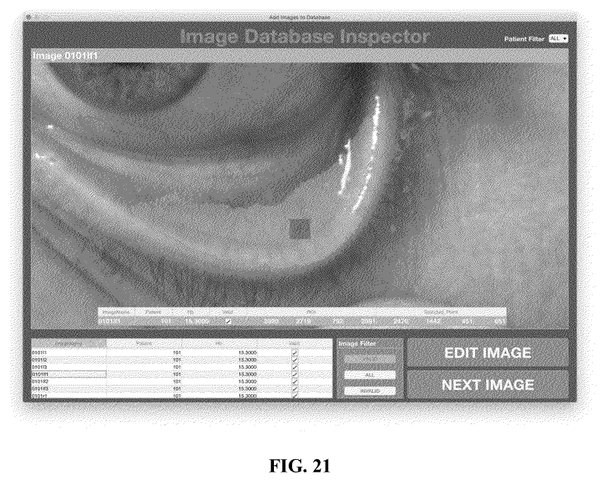

With respect to the image database (8-11), the production of an efficient Hb prediction model may requires repeated model development and testing. This process can be sped up through an exemplary Matlab application. The Matlab application may upload raw image files, request a seed point for region extraction and then store the information in a database. A user can interact with the Matlab application (e.g. upload raw image files, provide a seed point for region of extraction, etc.) via a graphical user interface (GUI) display screen as illustrated in FIG. 21. Each database entry can include a link to the raw file, extensive image metadata, and an image of the region of interest in high color resolution format. In one implementation, approximately 2000 images from 150 patients have been saved in a database. Information associated with the saved images (e.g., Hb measured by blood draw) can be used to generate a “look-up table”

With respect to feature extraction (11-14), each region of interest image in the database can vary in size (e.g., can have approximately 50,000 pixels). The color of each pixel can be defined by three numbers so that each image is stored as approximately 150,000 numbers in three 2-dimensional matrices. In addition, metadata (e.g., information from the camera used to capture the digital images) can be stored in the database. These metadata can provide access to information such as whether flash was used, exposure, or white balance.

High definition images containing large amounts of information in can be processed in an efficient manner, and features can be extracted from data to produce a set of parameters. These parameters can then be used to construct a mathematical model to predict Hb. For example, a parameter could be the average value of the red component of all pixels in the image.

In some implementations, 22 parameters for each image can encapsulate the information stored in the image itself as well as information from camera metadata. These 22 parameters can be generated for each image in the database creating a Parameter Table. Each row in the table may contain the 22 parameters for a specific image plus the measured Hb from blood draw used as the gold standard.

The aforementioned database can be used to generate and test new parameters easily and refine the parameter set, by testing the information content of each parameter. This can improve accuracy and efficiency. Ideal parameters can vary greatly with Hb levels while not being redundant. Redundancy can occur due to correlation between parameters. For example, average red value of an image can be a useful parameter but average green and blue levels can mirror red. Having all three as parameters therefore adds less information than one would expect and efficiency can be improved by dropping such redundant parameters.

Combinations of parameters can be synergistic. Image entropy can vary with flash so the flash parameter can be used in combination with entropy.

These interactions may be hard to predict one may need to continue to iteratively test the model to find the desirable (e.g., ideal) set of parameters that may give fast and accurate predictions.

With respect to the construction of the prediction model (14-16), based on the chosen parameters created from features extracted from each digital image a Parameter Table can be created. Each row can include a set of parameters for a given image as well as the actual measured Hb for that subject. The table can be used to construct mathematical models to predict Hb based on a given a set of parameters. For example, regression models including stepwise models can be used to reduce redundancy of parameters, and robust models that better tolerate noise in the data.

K fold testing can be used to created predictions for each image allowing error to be calculated when compared to measured Hb. 10 folds can be used in model testing. This means that 10 percent of the dataset can be randomly selected as the test set and the model is trained using the remaining 90%. The model is then tested with the 10% test set. This process is repeated to identify the best models and identify image properties which improve or worsen Hb prediction.

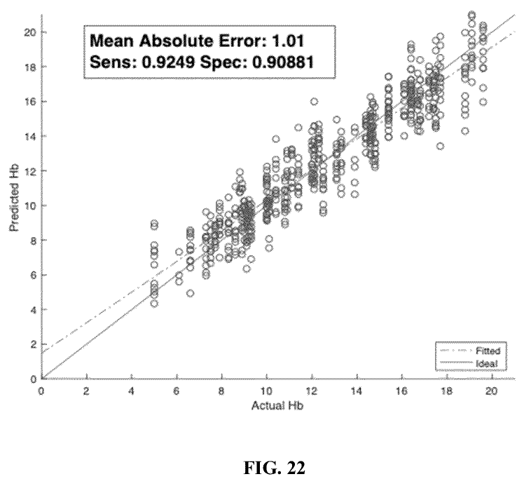

This approach can result in the following equation that is highly predictive of Hb concentration: Hb = 1 + B2 + R1 x G1 + R1 x HHR + R1 x ASN3 + B1 x HHR + B1 x ASN3 + B1 x BV + HHR x ASN1+ H x ASN1 + H x ASN3 + H x BV [Yi-Ming Chem et al. Examining palpebral conjunctiva for anemia assessment with image processing methods, https://pubmed.ncbi.nlm.nih.gov/28110719/) This approach can allow for sensitivities of 92% for anemia detection. FIG. 22 illustrates an exemplary plot of predicted vs actual Hb measurement across 120 patients. In some implementations, prediction of Hb concentration can be improved using neural network classification techniques. In some implementations, high accuracy has been achieved in the 10-15 g/dL range. Statistical corrections can be applied to the extremes of anemia where a loss of accuracy has been noted. This would extend the operating range of Hemacam from 6 to 18 g/dL. This would be facilitated after the real world testing data set (N=202) is collapsed into the present look up data set of 142 participants. Currently, mathematical models can be designed and tested in Matlab as this can allow for rapid prototyping and testing. The algorithm can be implemented in Swift for use in an application using Xcode development suite.

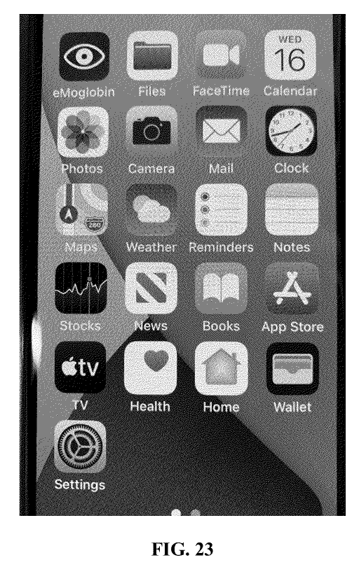

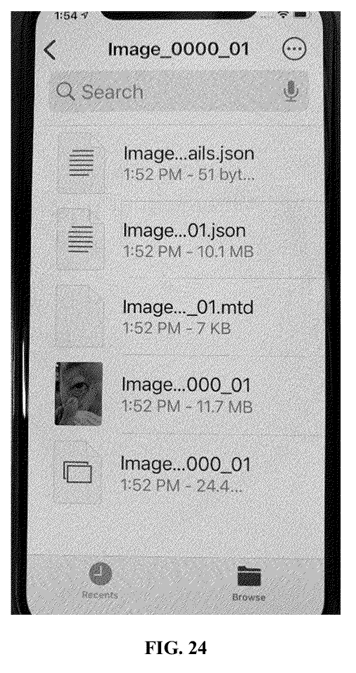

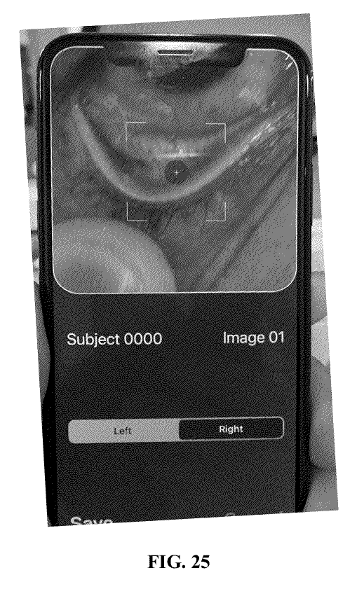

FIG. 23 illustrates an exemplary application (“eMoglobin”) installed on an iPhone. The application can allow for non-invasive detection of Hb (e.g., based on the method described in FIG. 17, FIG. 18, etc.). FIG. 24 illustrates an exemplary GUI of the application for capturing an image of the eye. The GUI can allow for auto focus during the capture of the image. After the image is captured, the application can identify an ROI, an autoselect relevant portions of the image. FIG. 25 illustrates an exemplary file folder for storing data for the application (e.g., captured images of the eye).

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

The subject matter described herein can be implemented in digital electronic circuitry, or in computer software, firmware, or hardware, including the structural means disclosed in this specification and structural equivalents thereof, or in combinations of them. The subject matter described herein can be implemented as one or more computer program products, such as one or more computer programs tangibly embodied in an information carrier (e.g., in a machine readable storage device), or embodied in a propagated signal, for execution by, or to control the operation of, data processing apparatus (e.g., a programmable processor, a computer, or multiple computers). A computer program (also known as a program, software, software application, or code) can be written in any form of programming language, including compiled or interpreted languages, and it can be deployed in any form, including as a stand alone program or as a module, component, subroutine, or other unit suitable for use in a computing environment. A computer program does not necessarily correspond to a file. A program can be stored in a portion of a file that holds other programs or data, in a single file dedicated to the program in question, or in multiple coordinated files (e.g., files that store one or more modules, sub programs, or portions of code). A computer program can be deployed to be executed on one computer or on multiple computers at one site or distributed across multiple sites and interconnected by a communication network.

The processes and logic flows described in this specification, including the method steps of the subject matter described herein, can be performed by one or more programmable processors executing one or more computer programs to perform functions of the subject matter described herein by operating on input data and generating output. The processes and logic flows can also be performed by, and apparatus of the subject matter described herein can be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processor of any kind of digital computer. Generally, a processor will receive instructions and data from a read only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. Information carriers suitable for embodying computer program instructions and data include all forms of non volatile memory, including by way of example semiconductor memory devices, (e.g., EPROM, EEPROM, and flash memory devices); magnetic disks, (e.g., internal hard disks or removable disks); magneto optical disks; and optical disks (e.g., CD and DVD disks). The processor and the memory can be supplemented by, or incorporated in, special purpose logic circuitry.

To provide for interaction with a user, the subject matter described herein can be implemented on a computer having a display device, e.g., a CRT (cathode ray tube) or LCD (liquid crystal display) monitor, for displaying information to the user and a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The techniques described herein can be implemented using one or more modules. As used herein, the term “module” refers to computing software, firmware, hardware, and/or various combinations thereof. At a minimum, however, modules are not to be interpreted as software that is not implemented on hardware, firmware, or recorded on a non-transitory processor readable recordable storage medium (i.e., modules are not software per se). Indeed “module” is to be interpreted to always include at least some physical, non-transitory hardware such as a part of a processor or computer. Two different modules can share the same physical hardware (e.g., two different modules can use the same processor and network interface). The modules described herein can be combined, integrated, separated, and/or duplicated to support various applications. Also, a function described herein as being performed at a particular module can be performed at one or more other modules and/or by one or more other devices instead of or in addition to the function performed at the particular module. Further, the modules can be implemented across multiple devices and/or other components local or remote to one another. Additionally, the modules can be moved from one device and added to another device, and/or can be included in both devices.

The subject matter described herein can be implemented in a computing system that includes a back end component (e.g., a data server), a middleware component (e.g., an application server), or a front end component (e.g., a client computer having a graphical user interface or a web interface through which a user can interact with an implementation of the subject matter described herein), or any combination of such back end, middleware, and front end components. The components of the system can be interconnected by any form or medium of digital data communication, e.g., a communication network. Examples of communication networks include a local area network (“LAN”) and a wide area network (“WAN”), e.g., the Internet.

Approximating language, as used herein throughout the specification and claims, may be applied to modify any quantitative representation that could permissibly vary without resulting in a change in the basic function to which it is related. Accordingly, a value modified by a term or terms, such as “about” and “substantially,” are not to be limited to the precise value specified. In at least some instances, the approximating language may correspond to the precision of an instrument for measuring the value. Here and throughout the specification and claims, range limitations may be combined and/or interchanged, such ranges are identified and include all the sub-ranges contained therein unless context or language indicates otherwise.

1-23. (canceled)

24. A method comprising:
turning a lower eyelid inside out to for exposure of a conjunctiva;
capturing a plurality of images of the exposed conjunctiva by a camera in a user device using image capture to minimize any motion, any shadow and any glare; and
processing selected regions of the captured images using an image analysis algorithm utilizing an on-device interactive application, the processing accounting for ambient lighting, the glare, and pigmentation of a surrounding skin, the processing comprising:
receiving data indicative of a user selection of a first pixel of a first image of the plurality of images representative of conjunctiva color;
determining a region of interest associated with the selected first pixel, the determining comprising identifying pixels adjacent to the first pixel having color parameter values within a predetermined range of a first color parameter value of the first pixel;
determining a first plurality of parameters associated with the region of interest;
generating a matrix comprising a first plurality of rows and a second plurality of columns, wherein the first plurality of rows are representative of the plurality of images and the second plurality of column are representative of the first plurality of parameters; and
performing a regression analysis on the matrix to generate a predictive model for hemoglobin.

25. The method of claim 24, wherein the on-device interactive application is configured to provide instructions to the user to reduce a motion of the camera capturing the images, maximize image focus and reducing shadow/glare on the exposed conjunctiva or guide them to a desirable region on the conjunctiva containing maximal vascularity.

26. The method of claim 24, wherein the regression analyses comprises machine learning and neural network paradigms.

27. The method of claim 24, further comprising manipulating the received data.

28. The method of claim 27, wherein the manipulating comprises generating a mapped data by mapping one or more values of the received data based on a predetermined look-up table.
